(19) United States
(12) Patent Application Publication
(10) Pub. No.: US 2009/0029482 A1
(43) Pub. Date: Jan. 29, 2009

(54) FUNCTIONAL PARTICLE, AND METHOD FOR SEPARATION OF TARGET SUBSTANCE USING THE SAME

(76) Inventors: Naoki Usuki, Osaka (JP); Masakazu Mitsunaga, Osaka (JP); Kenji Kohno, Osaka (JP); Hisao Kanzaki, Osaka (JP)

Correspondence Address: BIRCH STEWART KOLASCH & BIRCH, PO BOX 747, FALLS CHURCH, VA, 22040-0747, US

(21) Appl. No.: 12/281,596

(22) PCT Filed: Apr. 27, 2007
(86) PCT No.: PCT/JP2007/059550

§ 371 Date: Sep. 3, 2008

(51) Int. Cl. B01D 15/26; G01N 33/543; B01J 20/26; B32B 5/16

(52) U.S. Cl. 436/531; 428/402; 210/660; 502/402

(57) ABSTRACT

The particles of the present invention are high density particles which enable a preferential binding of a target substance thereto and inhibit a binding of “substances other than the target substance” thereto. The particles of the present invention are characterized in that a substance or functional group capable of binding to a target substance is immobilized on the surface of a particle body; the particles have a density of 3.5 g/cm³ to 9.0 g/cm³ and the particle body has no through-pore. The particles of the present invention are also characterized by a specific surface area of 0.0005 m²/g to 1.0 m²/g.

### TECHNICAL FIELD

The present invention relates to a functional particle suited for a separation, immobilization, analysis, extraction, purification, reaction or the like of a target substance. The present invention also relates to a method for treating a target substance by using of the particle.

### BACKGROUND OF THE INVENTION

Composite particles capable of specifically binding to or reacting with particular kinds of target substances have conventionally been well known as functional materials for biochemical applications. Examples of such applications include a quantitative determination, a separation, a purification, and an analysis of the target substances such as cells, proteins, nucleic acids and chemical substances. See Patent Document 1: Japanese Patent Kokai Publication No. 4-501956. The above composite particles are magnetic particles which are formed by incorporating a magnetic material into nonmagnetic beads. When the composite particles are used for separating target substances from a sample, the composite particles are supplied in the sample containing the target substances in order to allow the target substances to bind to the surfaces of composite particles. Subsequently, a magnetic field is applied in order to allow the composite particles to assemble and aggregate in the sample. By recovering the assembled and aggregated composite particles, the target substance together with the composite particles can be separated. This method uses the magnetic field or magnetism (the method using the magnetic field or magnetism hereinafter can be also referred to as “magnetic separation method”, or simply referred to as “magnetic separation”). Therefore, this method has such a feature that it can be carried out even if the amount of the sample is smaller than the amount used in a centrifugal separation method, a column separation method, an electrophoresis method or the like, and also can be carried out in a short time without causing denaturation of the target substances. However, the above composite particles have a small density of 1.0 g/cm³ to 3.4 g/cm³, which makes it difficult to achieve an efficient aggregation of the composite particles. The reason for the comparatively small density of the composite particles is that they are prepared from a low-density resin or silica serving as base material and a magnetic powder material dispersed therein. In other words, considering that the density of the composite particles depends on the amount of the magnetic powder material, the content of such magnetic powder material is only about 20% by weight at most when calculated from the magnetization amount, and therefore the density of the composite particles is close to the low density of the base material, i.e. the low-density resin or silica.

In contrast, an example using high-density zirconia particle is described in Patent Document 2: Japanese Patent Kokai Publication No. 9-503989. However, the zirconia particles described in Patent Document 2 are porous particles having a three-dimensional interpenetrating network (namely, through-pore) and thus a nonspecific binding phenomenon is likely to occur upon separation of the target substance. In other words, the substances other than the target substances tend to bind to the zirconia particles, and thus the target substances are hard to preferentially bind to the particles, which will prevent an achievement of the separation of the target substances. Furthermore, the zirconia particles described in Patent Document 2 are porous particles and thus tend to incorporate an entrained gas (e.g. air) upon supplying the particles into the sample containing the target substances. As a result, the buoyancy of the incorporated gas prevents the movement and aggregation of the particles in the sample, which leads to an unsatisfactory separation of the target substances (namely, the time required for separation of the target substances is prolonged).

### DISCLOSURE OF THE INVENTION

Under these circumstances, the present invention has been created. In other words, an object of the present invention is to provide particles which are suited for separation of a target substance from the standpoint of not only a movement and aggregation of the particles but also a nonspecific binding. Another object of the present invention is to provide a method for separating a target substance by the use of the above particles, or a method for obtaining particles with a target substance immobilized thereon. A yet another object of the present invention is to provide a method for performing an analysis, extraction, purification or reaction of a target substance by the use of the particles of the present invention.

In order to achieve the above objects, the present invention provides particles to which a target substance can bind, characterized in that;

“substance or functional group capable of binding to a target substance” is immobilized on the surface of each of their particle bodies;

the particles have a density of 3.5 g/cm³ to 9.0 g/cm³; and

each of the particle bodies has no through-pore (through-hole).

The particles of the present invention have “substance or functional group capable of binding to a target substance” immobilized thereon. In other words, “substance or functional group to which a target substance can bind” is immobilized. Therefore, when the target substance and particles coexist with each other, the target substance can bind to the particles. Therefore, the particles of the present invention can be used for not only various applications such as separation, purification and extraction of the target substance, but also applications of tailor-made medical technologies. As used in this description and claims, “target substance” substantially means an object substance in various applications such as separation, extraction, quantitative determination, purification and analysis. “Target substance” may be any suitable substances as long as it can bind to the particles directly or indirectly. Examples of the target substance include nucleic acids, proteins (e.g. avidin, biotinylated HRP and the like), sugars, lipids, peptides, cells, eumycetes (fungus), bacteria, yeasts, viruses, glycolipids, glycoproteins, complexes, inorganic substances, vectors, low molecular compounds, high molecular compounds, antibodies and antigens. The particles of the present invention have various functions, considering that they can be used for separation, purification, extraction and analysis of various target substances. It should be therefore noted that the particles of the present invention can be called “functional particles”.

The particles of the present invention are characterized by the density of 3.5 g/cm³ to 9.0 g/cm³, and thus the density (or specific gravity) higher than that of particles used commonly for separation of the target substance. The particles of the present invention are also characterized in that they are not in porous form in light of the fact that through-pores are not formed in the bodies of the particles. In this regard, the particles have a comparatively small specific surface area of 0.0005 m²/g to 1.0 m²/g.

As used in this description and claims, the expression “particle body has no through-pore” means that the body of the particle is substantially solid and thus the particle has no “interpenetrating network structure”. That is to say, the phrase “particle body having no through-pore” has the same meaning as “particle body or core portion thereof is solid”, “even if the particle has a rough surface, no recess exists in the interior of the particle” and “the bulk density of the particle is higher as compared with that of a conventional porous particle”.

In the present invention, there is also provided a method for separating a target substance by the use of the above-mentioned particles. This invention relates to a method for separating a target substance from a sample or obtaining particles with a target substance immobilized thereon, by the use of the particles of the present invention, the method comprising the steps of:

(i) bringing the particles and the sample containing the target substance into contact with each other, and thereby binding the particles and the target substance to each other;
(ii) allowing the sample to stand, and thereby allowing a spontaneous sedimentation of the particles in the sample; and
(iii) recovering the particles precipitated in the sample, and thereby separating the target substance from the sample or obtaining the particles with the target substance immobilized thereon.

The method of the present invention is characterized in that the particles having the target substance which has bound thereto are assembled and aggregated by a spontaneous sedimentation thereof. In other words, the method of the present invention is characterized in that a magnetic field or magnetism is not employed for a movement and aggregation of the particles. The separation of the target substance can be achieved only by a spontaneous sedimentation of the particles. This is due to a higher spontaneous sedimentation rate of the particles as compared with that of the prior art.

The particles of the present invention not only have a high density of 3.5 g/cm³ to 9.0 g/cm³ but also have no through-pore, and thus they have smaller specific surface area of 0.0005 m²/g to 1.0 m²/g. This means that a movement rate attributable to the spontaneous sedimentation of the particles can lead to a sufficient separation rate even when a centrifugal separation method or a magnetic separation method is not employed. In other words, the particles of the present invention are suited for the spontaneous sedimentation in terms of not only their density but also their specific surface area. As for the porous particles having a large specific surface area, “specific surface area” means that the particles include lots of voids therein. When such porous particles are supplied to a sample containing a target substance, they tend to incorporate an entrained gas (e.g. air) into the particles, and thereby the buoyancy attributable to the incorporated gas causes a decrease in a sedimentation rate. In contrast, the particles of the present invention have no through-pore and thus are substantially non-porous particles in terms of a specific surface area of 0.0005 m²/g to 1.0 m²/g, which will lead to the alleviation of an adverse influence of the gas as described above.

As used in this description and claims, the phrase “spontaneous sedimentation (natural sedimentation)” means that particles settle out in a liquid by gravitation. As used in this description and claims, the term “separation” means a separation of a target substance from a sample which contains the target substance. Examples of the target substance include nucleic acids, proteins, sugars, lipids, peptides, cells, eumycetes (fungus), bacteria, yeasts, viruses, glycolipids, glycoproteins, complexes, inorganic substances, vectors, low molecular compounds, high molecular compounds, antibodies and antigens. Examples of the sample include body fluids such as urine, blood, serum, plasma, sperm, saliva, sweat, tears, ascitic fluids and amniotic fluids from humans or animals; suspension liquids, extraction liquids, solutions and crushed solutions of organs, hair, skin, nail, bone, muscle and nervous tissue from humans or animals; suspension liquids, extraction liquids, solutions and crushed solutions of stools; suspension liquids, extraction liquids, solutions and crushed solutions of cultured cells or cultured tissues; suspension liquids, extraction liquids, solutions and crushed solutions of viruses; suspension liquids, extraction liquids, solutions and crushed solutions of fungus bodies; suspension liquids, extraction liquids, solutions and crushed solutions of soil; suspension liquids, extraction liquids, solutions and crushed solutions of plants; suspension liquids, extraction liquids, solutions and crushed solutions of food and processed food; and drainage water. More specifically, term “separation” substantially means that a target substance contained in a sample is allowed to bind to the particles and the target substance is separated from the sample by allowing the target substance-binding particles to move. The phrase “separation rate” substantially means a rate of the particle movement in the sample wherein the particles have the target substance which has bound thereto. In a case of “spontaneous sedimentation”, the phrase “separation rate” substantially means a sedimentation rate of particles. The high separation rate can reduce the time required for separating the target substance from the sample. In a case where the particles of the present invention are magnetic particles, the separation rate can be additionally increased by applying a magnetic field thereto.

The spontaneous sedimentation of the particles of the present invention contributes to a satisfactory separation rate. This means that the use of the particles of the present invention enables simplicity of a separation, immobilization, analysis, extraction, purification or reaction of the target substance. Namely, the use of the particles of the present invention can provide a simple system for performing separation, immobilization, analysis, extraction, purification or reaction of the target substance. In addition, the particles of the present invention are effective for miniaturization or chip processing of the system.

Since the particles of the present invention not only have a high density but also can be substantially regarded as non-porous particles in terms of a smaller specific surface area of 0.0005 m²/g to 1.0 m²/g, it is possible to suppress a nonspecific binding phenomenon in which “substances other than the target substance” bind to the particles. Due to the non-porosity of the particles, the particles have a small proportion of particle pores or particle surfaces capable of absorbing and adsorbing “substances other than the target substance”. Alternatively, there is substantially no particle pore or particle surface capable of absorbing and adsorbing “substances other than the target substance” in the particles. As a result, the binding of “substances other than the target substance” to the particles is prevented.

Based on the suppressed nonspecific binding phenomenon, the purification or separation of the target substance can be efficiently carried out by a simple operation.

On the body surface of each particle of the present invention, a polymer may be present. In this case, “substance or functional group capable of binding to a target substance” can be immobilized on the surface of the polymer (hereinafter also referred to as “coating polymer”). The use of the coating polymer makes it possible to immobilize “substance or functional group capable of binding to a target substance” on the surface of the particles even when it is difficult for “substance or functional group capable of binding to a target substance” to covalently bond with the particle body. In an application where the immobilized “substance or functional group capable of binding to a target substance” tends to separate from the surface of the particle body due to various conditions, such separation can be prevented by immobilizing “substance or functional group capable of binding to a target substance” on the coating polymer. In a case where, as the coating polymer, a polymer which prevents a penetration of various molecules or metal ions therethrough is selected, an elution of ions from the surface of the particle body or the inside of the particles can be suppressed (namely, metal ions generated from a constituent component of particles is prevented from being eluted). In this case, an unnecessary reactions caused by metal ions in various applications of the particles can be also suppressed.

As described above, the particles used in the separation method of the present invention are high density particles, and substantially regarded as non-porous particles, namely, they have a comparatively small specific surface area of 0.0005 m²/g to 1.0 m²/g. As a result, the incorporation of the entrained gas (e.g. air) into the particles is suppressed upon supplying the particles into a sample containing a target substance, which leads to an achievement of the sufficient separation rate only by the spontaneous sedimentation of the particles. Furthermore, as described above, the particles used in the separation method of the present invention can suppress “nonspecific binding phenomenon in which substances other than a target substance bind to the particles”. Therefore, even when the sample contains “substances other than a target substance”, the separation method of the present invention enables the target substance to preferentially bind to the particles, and thereby the target substance can be efficiently separated.

### BRIEF DESCRIPTION OF THE DRAWINGS

FIG. 1 schematically illustrates the steps of a method of the present invention.

FIG. 2 is a micrograph showing non-porous yttrium-doped zirconia particle p₁ being a precursor particle of Example 1.

FIG. 3 is an enlarged micrograph showing a surface of non-porous yttrium-doped zirconia particle p₁ being a precursor particle of Example 1.

FIG. 4 is a micrograph showing porous silica particles r₆ being precursor particles of Comparative Example 6.

FIG. 5 is an enlarged micrograph showing a surface of porous silica particle r₆ being a precursor particle of Comparative Example 6.

In the figures, reference numerals mean the following elements:
1 . . . particle(s) of the present invention
2 . . . target substance(s)
3 . . . substance(s) other than the target substance(s)
4 . . . sample

### BEST MODES FOR CARRYING OUT THE INVENTION

First, particles of the present invention will be described, and then a separation method of the present invention will be described.

The particles of the present invention have a density suited for separation of a target substance. That is, the particles of the present invention have a density enabling a comparatively high sedimentation rate of the particles when the particles are dispersed in samples, for example, body fluids such as urine, blood, serum, plasma, sperm, saliva, sweat, tears, ascitic fluid and amniotic liquid of humans or animals; suspension liquids, extraction liquids, solutions or crushed solutions of organs, hair, skin, nail, bone, muscle or nervous tissue of humans or animals; suspension liquids, extraction liquids, solutions or crushed solutions of stools; suspensions liquid, extraction liquids, solutions or crushed solution of cultured cells or cultured tissues; suspension liquids, extraction liquids, solutions or crushed solutions of virus; suspension liquids, extraction liquids, solutions or crushed solutions of fungus bodies; suspension liquids, extraction liquids, solutions or crushed solutions of soil; suspension liquids, extraction liquids, solutions or crushed solutions of plants; suspension liquids, extraction liquids, solutions, or crushed solutions of food and processed food; or drainage water. When the density of the particles is less than 3.5 g/cm³, only the spontaneous sedimentation of the particles will not bring about a preferable movement rate thereof from a practical standpoint. In contrast, the particle density of more than 9.0 g/cm³ is not preferred for a stirring operation upon binding of the target substance. In this regard, the density of the particles of the present invention is in the range of 3.5 g/cm³ to 9.0 g/cm³, preferably in the range of 5.0 g/cm³ to 8.0 g/cm³, and more preferably in the range of 5.5 g/cm³ to 7.0 g/cm³. As used in this description and claims, the term “density” means a true density (real density) in which only a volume occupied by the substances is used as a volume for calculation of density, and such density can be determined by a true density measuring device ULTRAPICNOMETER 1000 (manufactured by Yuasa Ionics Inc.).

The specific surface area of the particles of the present invention is preferably in the range of 0.0005 m²/g to 1.0 m²/g, more preferably in the range of 0.005 m²/g to 0.5 m²/g, still more preferably in the range of 0.01 m²/g to 0.2 m²/g, for example 0.01 m²/g to 0.05 m²/g. Therefore, the particles of the present invention are substantially regarded as non-porous particles, and thus the possibility of “substances other than a target substance” binding to the particles (namely, “nonspecific binding”) is suppressed. This means that the accuracy of the separation of the target substance is improved. Since the particles of the present invention are substantially non-porous particles and include no through-pore (namely, interpenetrating network structure) therein, the incorporation of the entrained gas (e.g. air) into the particles is suppressed upon supplying the particles into a sample containing a target substance. As a result, a sufficient separation rate can be achieved only by spontaneous sedimentation of the particles.

“Specific surface area” as used in this description and claims is a specific surface area determined by a specific surface area pore distribution analyzer SA3100 (manufactured by Coulter Co.).

As described above, it is possible to achieve a satisfactory separation rate only by the spontaneous sedimentation of the particles. In other words, a spontaneous sedimentation rate of the particles of the present invention is high in a sample containing a target substance.

The material of the particle body is not limited as long as the particles of the present invention have the above-described density and specific surface area. For example, it is preferred that the particle body is formed of a metal or metal oxide. More specifically, the particle body is formed of at least one kind of a material selected from the group consisting of zirconia (zirconium oxide, yttrium-doped zirconium oxide), iron oxide, alumina, nickel, cobalt, iron, copper and aluminum.

It is beneficial that the particles of the present invention are magnetized (hereinafter, the magnetized particles of the present invention are referred to as “magnetic particles”) since an auxiliary magnetic separation can be additionally applied to the spontaneous sedimentation of the particles. When the auxiliary magnetic separation is additionally applied, the particles are allowed to move more quickly, which will lead to a shorter time required for separating a target substance (more specifically a shorter time required for separating “target substance which have bound to the particles”). Further, a pipetting or decantation operation can be easily performed by collecting or settling the particles by means of magnetism.

In a case where the coating polymer is present on the surface of a particle body, it is usually difficult to impart magnetism to the coating polymer. It is therefore preferred that a magnetized particle is used as the body of the particle.

The material for the bodies of the magnetic particles is not limited as long as the particles are magnetized. For example, it is preferred that the bodies of the magnetic particles are formed of at least one kind of iron oxide selected from the group consisting of a garnet-structured oxide comprising a transition metal and an iron, ferrite, magnetite, and γ-iron oxide. Alternatively, the bodies of the magnetic particles may contain at least one kind of metallic material selected from the group consisting of nickel, cobalt, iron and alloy thereof. As used herein, “garnet-structured oxide comprising a transition metal and an iron” is generally referred to as YIG. For example, “garnet-structured oxide comprising a transition metal and an iron” is a compound represented by the composition formula of Y₃Fe₅O₁₂, or BiₓY₃₋ₓFe₅O₁₂ (0<X<3) in which a portion of Y in the compound is substituted with bismuth.

Alternately, the magnetic particles may be formed by coating non-magnetized particles with a magnetic substance, or may be formed by simply providing a magnetic substance on the non-magnetized particles. In this regard, an electroless plating process, electroplating process, sputtering process, vacuum deposition process, ion plating process or chemical deposition process can be employed. Examples of “non-magnetized particles” as used herein include high density particles formed of zirconia (zirconium oxide, yttrium-doped zirconium oxide), alumina or the like. When the content of the high-density magnetic substance is higher, lower-density particles formed of aluminum, silica or resin can be also used. Examples of “magnetic substance” used for coating a part of surface or whole surface of the particle body include iron oxides such as ferrite, magnetite, γ-iron oxide and garnet-structured oxide comprising a transition metal and an iron, which are similar to the above-described material for the bodies of the magnetic particles. Nickel, cobalt, iron or alloy thereof also may be used for the magnetic substance.

If the amount of the magnetic substance for coating the surfaces of the non-magnetized particles is too small, the intensity of the magnetization of the particles decreases. This is not preferred for magnetic separation. It is therefore preferred that the volume of the coating magnetic substance accounts for 5% or more of the volume of particles (i.e. particles with the coating magnetic substance thereon). As for a thickness of the coating magnetic substance of each particle, it is preferred that such thickness accounts for 1.7% or more of the diameter of each particle (i.e. particle with the coating magnetic substance thereon). It should be noted that not only an embodiment wherein the coating magnetic substance is formed on “non-magnetized particles”, but also an embodiment wherein a magnetic substance is included inside “non-magnetized particles” is possible.

Magnetic characteristics of magnetic particles include, for example, “saturation magnetization” and “coercive force (coercitivity)”. As the value of the saturation magnetization increases, the responsiveness of particles to magnetic field is generally improved. In order to magnetize the particles having a comparatively high density, it is necessary to supply a magnetic substance on the surface of or in the non-magnetized particles. In this regard, the magnetic substance has density smaller than that of the non-magnetized particles, and thus the required density must be achieved by restricting the amount of the magnetic substance to be supplied. When the particle body is coated with the non-magnetic polymer, it is actually difficult to achieve a saturation magnetization higher than that of particles formed of only the magnetic substance. In other words, it is actually difficult to achieve a saturation magnetization more than 85 A·m²/kg. In contrast, when the saturation magnetization is less than 0.5 A·m²/kg, the responsiveness of the particles to the magnetic field falls below a required level and thus it is not preferred. Therefore, the saturation magnetization of the particles of the present invention is preferably in the range of 0.5 A·m²/kg to 85 A·m²/kg (0.5 emu/g to 85 emu/g), more preferably in the range of 3 A·m²/kg to 10 A·m²/kg (3 emu/g to 10 emu/g), for example 4 A·m²/kg to 7 A·m²/kg (4 emu/g to 7 emu/g). When the value of the coercive force increases, the particles tend to aggregate. However, when the value of the coercive force is too large, the dispersion of the particles is inhibited due to an excessively strong aggregation action. Namely, too large coercive force is not preferred in terms of the binding of the target substance. Therefore, the coercive force is preferably in the range of 0 kA/m to 23KA/m (0 to 300 Oe), more preferably in the range of 0 kA/m to 15.95 kA/m (0 to 200 Oe), and still more preferably in the range of 0 kA/m to 7.97 kA/m (0 to 100 Oe).

The values of “saturation magnetization” and “coercive force” as used in this description and claims are values measured by a vibration sample type magnetometer (manufactured by TOEI INDUSTRY CO., LTD., Model VSM-5). Specifically, the value of “saturation magnetization” is a value determined from the magnetization amount when the magnetic field of 797 kA/m (10 kOe) is applied. The value of “coercive force” is a value of the applied magnetic field at which the magnetization amount becomes zero when the magnetic field is returned to zero after applying the magnetic field of 797 kA/m, and then the magnetic field is gradually increased in the reverse direction.

There is no restriction on the shape of particles of the present invention. Each shape of the particles may be sphere, ellipsoid, granule, plate, needle or polyhedron (e.g. cube). In order to decrease variation between particles in terms of the binding of the target substance thereto, each shape of the particles is preferably a regular shape, and more preferably a spherical shape. In a case where the coating magnetic substance is provided on the body of the non-magnetized particle, it is preferred that “body of non-magnetized particle” has a spherical or ellipsoidal shape.

It is preferred that the particles of the present invention have an average size (namely, “average particle size”) of 1 μm to 1 mm. When the average particle size is less than 1 μm, it becomes difficult to sufficiently increase the particle movement rate attributable to spontaneous sedimentation upon separating the target substance. In contrast, when the average particle size is more than 1 mm, the sedimentation of the particles is completed before the biding of the target substance thereto, which will lead to an unsatisfactory separation of the target substance. The average particle size is more preferably in the range of 5 μm to 500 μm, and still more preferably in the range of 10 μm to 100 μm. As used in this description, the phrase “particle size” substantially means a maximum length among lengths in all directions of each particle (lengths including a thickness of the coating polymer in a case where the polymer is provided on the particle body). As used in this description and claims, the phrase “average particle size” (i.e. “average size of particles”) substantially means a particle size calculated as a number average by measuring each size of 300 particles for example, based on an electron micrograph or optical micrograph of the particles. As the size of particles formed of pure metal becomes smaller, a rapid oxidation may occur, and thereby an ignition of the particles may also occur. In this regard, the comparatively large particle size of the particles according to the present invention contributes to the prevention of the rapid oxidation and ignition of the particles.

It is preferred that “substance capable of binding to the target substance” (hereinafter also referred to as “substance to which a target substance can bind”) immobilized on the body surface of each particle of the present invention is at least one kind of a substance selected from the group consisting of biotin, avidin, streptavidin and neutrAvidin. It is preferred that “functional group capable of binding to the target substance” (hereinafter also referred to as “functional group to which a target substance can bind”) immobilized on the body surface of each particle of the present invention is at least one kind of a functional group selected from the group consisting of carboxyl group, hydroxyl group, epoxy group, tosyl group, succinimide group, maleimide group, thiol group, thioether group, sulfide functional group (e.g. disulfide group), aldehyde group, azido group, hydrazide group, primary amino group, secondary amino group, tertiary amino group, imide ester group, carbodiimide group, isocyanate group, iodoacetyl group, halogen-substitution of carboxyl group and double bond. “Functional group to which a target substance can bind” may be derivatives of these functional groups.

As used in this description and claims, the term “immobilization (immobilized)” substantially means an embodiment wherein “substance to which a target substance can bind” or “functional group to which a target substance can bind” exists in the vicinity of the surface of each particle body. Namely, the term “immobilization (immobilized)” does not necessarily mean only the embodiment wherein “substance to which a target substance can bind” or “functional group to which a target substance can bind” is directly attached to the surface of each particle body. Also, the term “immobilization (immobilized)” substantially means an embodiment wherein “substance or functional group to which a target substance can bind” is immobilized on at least a part of each particle surface. Accordingly, “substance or functional group to which a target substance can bind” is not necessarily immobilized over the entire surface of each particle. In a preferred embodiment, “substance or functional group to which a target substance can bind” is present on the entire surface of each particle so that each particle body is surrounded by “substance or functional group to which a target substance can bind”. As used in this description and claims, the expression “target substance binds” includes not only an embodiment wherein a target substance is “adsorbed” or “absorbed” to particles, but also an embodiment wherein a target substance binds to particles due to various kinds of “affinities” acting between the target substance and the particles.

According to the present invention, due to the fact that “substance to which a target substance can bind” or “functional group to which a target substance can bind” is immobilized on the body of each particle, the target substance can bind to the particle via “substance or functional group to which a target substance can bind”.

There is no restriction on the method for immobilizing “substance to which a target substance can bind” on the particle body. That is to say, any suitable methods may be used as long as the binding or adhering of “substance to which a target substance can bind” to the body of each particle is achieved. It is not necessarily the case that “substance to which a target substance can bind” is directly bound or adhered to the particle body. If necessary, the immobilization of “substance to which a target substance can bind” on the particles may be facilitated by adhering or introducing other substances, for example, a silicon-containing substance (e.g. siloxane, silane coupling agent and sodium silicate) or a resin having a functional group to which a target substance can bind or adhere, to the body of the particle in advance. Alternatively, a noble metal may be provided on the surface of the particle, followed by the adhering or introducing of other substances such as a sulfur-containing compound having a functional group to which a target substance can bind or adhere. In a case where the silicon-containing substance is used, the silicon-containing substance and the immobilized “substance to which a target substance can bind” are present on the surface of the particle body.

Just as an example, a silane coupling agent having an epoxy group or an amino group may be introduced to the surface of the particle body through a reaction so as to immobilize “substance to which a target substance can bind” on the surface of the particle body.

As with the immobilization of “substance to which a target substance can bind”, there is no restriction on the method for immobilizing “functional group to which a target substance can bind” on the particle body. That is to say, any suitable methods may be used as long as the binding or adhering of “functional group to which a target substance can bind” to the body of the particle is achieved. If necessary, “functional group to which a target substance can bind” may be converted into another functional group by a chemical treatment, and thereby its reactivity or adsorptivity is changed. As with the case of “substance to which a target substance can bind”, it is not necessarily the case that “functional group to which a target substance can bind” is directly bound or adhered to the particle body. If necessary, the immobilization of “functional group to which a target substance can bind” on the particles may be facilitated by adhering or introducing other substances, for example, a silicon-containing substance (e.g. siloxane, silane coupling agent and sodium silicate) or a resin having a functional group to which a target substance can bind or adhere, to the body of the particle in advance. Alternatively, a noble metal may be provided on the surface of the particle, followed by the adhering or introducing of other substances such as a sulfur-containing compound having a functional group to which a target substance can bind or adhere. In a case where a silicon-containing substance is used, the silicon-containing substance and the immobilized “functional group to which a target substance can bind” are present on the surface of the particle body.

Hereinafter, a method by the use of siloxane will be described as an example of the method for immobilizing “functional group to which a target substance can bind” on the bodies of the particles.

### Immobilization of Functional Group by the Use of Siloxane

In this immobilization, the surface of the particle body is preliminary coated with 1,3,5,7-tetramethylcyclotetrasiloxane (hereinafter abbreviated to “TMCTS”). This immobilization has a feature that the reaction is terminated at some point in time when the formation of a single-layered TMCTS film on the surface of each particle body is completed.

### (Pretreatment Step)

First, TMCTS is added to a dispersion liquid obtained by dispersing precursor particles into an organic solvent. In this regard, a sufficient amount of TMCTS is added so that a single-layered TMCTS film is formed on the surface of each particle. The resulting dispersion liquid is evaporated to remove a solvent therefrom, and particles are dried by heating them in a vacuum desiccator. Subsequently, the particles are heated in a thermostatic bath at 150° C. As the organic solvent, any suitable organic solvents may be used as long as they have a low boiling point which enables them to evaporate easily in an evaporator. Examples of the organic solvent include toluene, hexane, benzene and the like. When the inside of the vacuum desiccator is heated, the same effect as in case of a chemical vapor deposition method (CVD method) is provided, and thus it is necessary to control the heating temperature so as not to cause a decomposition of the evaporated TMCTS. Specifically, the heating temperature is preferably in the range of about 30 to 80° C. By heating the particles in the thermostatic bath at 150° C., the reaction between TMCTS(s) which have been adhered on the surface of particles is promoted. In this regard, when the temperature increases too much, the decomposition of TMCTS is likely to occur. Further, when the reaction is carried out for a long time, a reactive site required for the subsequent immobilization step of the functional group disappear. Therefore, the heating temperature is preferably in the range of 100° C. to 200° C. and the reaction time is preferably within 2 hours. The fact that the particles become hydrophobic implies that a TMCTS film has been formed on the surface of each particle.

### (Immobilization Step of Functional Group)

Subsequent to the pretreatment step, an immobilization step of a functional group is carried out. As a compound having a functional group to be immobilized, it is required to comprise a double bond at the terminal thereof, but there is no other restriction thereon. For example, the compound to be used may have any structures between a functional group and a double bond site. The functional group may be used alone, or a plurality of functional groups may be used. Plural kinds of functional groups may be also immobilized.

In a reaction process wherein a functional group such as an epoxy group or a carboxyl group is immobilized, the Si—H moiety contained in TMCTS and a double bond site of a compound having a functional group such as an epoxy group or a carboxyl group are reacted with each other, and thereby the functional group is introduced to the surface of particles. To this end, for example, the particles obtained in the pretreatment step are dispersed into a solvent, and then a catalyst and a compound having a functional group to be immobilized are added to the resulting dispersion liquid in a heated state. The resulting mixture is subject to a reaction for several hours. As the organic solvent, any suitable solvents may be used as long as the compound having a functional group to be immobilized can dissolve therein and a stable reaction rate is provided even when heated at 60° C. or higher. Examples of the organic solvent include water and ethylene glycol. Similarly, as the catalyst, any suitable catalysts may be used as long as they promote the above-described reaction. For example, chloroplatinic acid may be used.

Hereinafter, an embodiment of “particles in which a coating of polymer is provided on the particle body” will be described.

In a preferred embodiment, a coating of a polymer is provided on a part of the surface of each particle body, and “substance or functional group capable of binding to a target substance” is immobilized on the surface of the particle body or polymer. In another preferred embodiment, the entire surface of the particle body is coated with a polymer, and “substance or functional group capable of binding to a target substance” is immobilized on the surface of the polymer. In a case where the entire surface of the particle body is coated with a polymer, the particles of the present invention can be also referred to as “inclusion particles” or “particles having a core-shell structure” based on the form of the particles.

As a coating polymer provided on the surface of the particle body, a polymers which contribute to the immobilization of “substance to which a target substance can bind” or “functional group to which a target substance can bind” is preferred. In this case, the coating polymer can be selected on the basis of the kind of “substance to which a target substance can bind” or “functional group to which a target substance can bind”, conditions of use for particles, or other required characteristics of the particles. The representative examples of the coating polymer include at least one kind of synthetic polymer compound selected from the group consisting of polystyrene or derivatives thereof, poly(meth)acrylic acid, poly(meth)acrylic acid ester, polyvinylether, polyurethane, polyamide, polyvinyl acetate, polyvinyl alcohol, polyallylamine, and polyethyleneimine. The polymer is not limited to such synthetic polymer compound and may be a modified polymer or a copolymer thereof. Furthermore, for example, a semi-synthetic polymer compound such as hydroxyalkyl cellulose, carboxyalkyl cellulose and sodium alginate; or a natural polymer compound such as chitosan, chitin, starch, gelatin and gum arabic may be used. Still furthermore, a polymer having a functional group introduced thereto in advance may be used, wherein “substance or functional group capable of binding to a target substance” can bind and adhere to such functional group.

In a case where the main objective is to suppress a elution of metal ions (namely, ions of metal constituting a particle body) from the surface or inside of particles, the coating polymer capable of hindering the penetration of the various molecules or metal ions constituting the particle body may be used. When the particles is intended for the use in an aqueous system, the coating polymer capable of hindering a penetration of water may be used, and in this case, polystyrene, alkyl polymethacrylate, polyvinylether or polyvinyl acetate can be used, for example.

As used in this description and claims, the expression “coating . . . provided” substantially means an embodiment wherein a polymer adheres or exists on at least a part of the surface of particle, and a polymer may not necessarily adhere or exist on the entire surface of the particle body. In a preferred embodiment, the entire surface of the particle body is coated with a polymer so that the particle body is surrounded by a polymer film. This embodiment provides a beneficial effect in that the amount of “substance or functional group capable of binding to a target substance” to be immobilized on the surface of the polymer is increased, and the elution of the metal ions attributable to the constituent materials of the particle body is suppressed to a greater degree (namely, the elution of the ions of the metal constituting a particle body is suppressed).

There is no restriction on a method for providing the coating polymer on a particle body. That is to say, any suitable methods may be used as long as the polymer is attached to the surface of the particle body. For example, the following methods may be used:
(1) a method of initiating polymerization from the surfaces of precursor particles;
(2) a method of depositing a polymer on the surfaces of precursor particles by performing a polymerization under the presence of precursor particles;
(3) a method of polymerizing through enclosing precursor particles in a monomer emulsion; and
(4) a method of mixing a solution of a preliminarily polymerized polymer with precursor particles, and thereby depositing the polymer on the surface of the precursor particles.

The above methods will be described in more detail. With respect to the method (1), the coating polymer is provided on the surface of the precursor particles by binding or adsorbing an initiator and a chain transfer agent on the surface of precursor particles, followed by extending the polymer from the surface of the particles. With respect to the method (2), the coating polymer is provided on the surface of the precursor particles by performing a polymerization under the presence of precursor particles by the use of a monomer capable of depositing as the polymerization reaction proceeds. Such provision of the polymer can be efficiently performed by selecting electric charges of the polymer and the particles so as to attract them to each other or by immobilizing a polymerizable double bond on the surfaces of the particles. With respect to the method (3), a combination of a solvent and a monomer capable of forming a monomer emulsion therefrom is selected and precursor particles are included within such monomer emulsion. To this end, a polymerization is carried out so as to provide the coating polymer on the surfaces of the precursor particles. In this method, a surface treatment or surfactant for improving affinity with the monomer may be used so that precursor particles preferentially exist in the monomer emulsion. With respect to the method (4), the coating polymer is provided on the surface of the precursor particles by incorporating the precursor particles into a polymer solution, followed by decreasing the solubility of the polymer and thus depositing the polymer through adding a poor solvent, varying the pH or adding a large amount of a salt. In this method, the provision of the polymer can be efficiently performed by selecting electric charges of the polymer and the particles so as to attract them to each other or by immobilizing a polymerizable double bond on the surfaces of the particles. Also, the precursor particles may be alternately immersed in polymer solutions each having different electric charge to form a lamination layer(s) on the surfaces of the particles.

In the above-described methods, various techniques such as a microencapsulation and an emulsion polymerization, which have conventionally been known, are available.

Prior to the provision of the coating polymer, the surfaces of precursor particles may be subjected to a particular treatment. Examples of such treatments include a magnetization treatment, a coating treatment with a metal or an inorganic substance, an adsorption treatment with a surfactant, a treatment with a reactive substance such as a silane coupling agent or a titanium coupling agent, a siloxane coating treatment, a treatment for introducing a functional group to Si—H of siloxane (hydrosilylation reaction), an acid treatment or alkali treatment, a solvent washing treatment, a polishing treatment and the like. These treatments contribute to a removal of stains from the surfaces of precursor particles, a control of electric charge for the surfaces of precursor particles, or an introduction of a reactive functional group to the surface of particles, which will lead to an improvement of the provision of the coating polymer or the adhesion between the coating polymer and the surfaces of particles. In a case where the silicon-containing substance (e.g. siloxane or silane coupling agent) is used, it should be understood that, in addition to “substance or functional group to which a target substance can bind” and the coating polymer, such silicon-containing substance exists on the body surfaces of the particles of the present invention. For example, the silicone-containing compound may exist between the surface of the particle body and the surface of the coating polymer. By preliminarily attaching or adsorbing an initiator and/or a polymerizable double bond onto the surface of precursor particle, the polymer is likely to deposit on the surface of the particle upon polymerization. This is effective for providing the coating polymer on the surfaces of the particles. In addition, it is possible to employ other processes to give other effects such as a reduction effect of nonspecific binding, a suppression effect of elution of metal ions, an adjustment effect of density and an imparted effect of color and fluorescence.

The coating polymer may be subjected to a crosslinking treatment. When the coating polymer is crosslinked, characteristics such as durability, solvent resistance and low swelling of the coating polymer can be improved. There is no restriction on a method for forming the crosslinked polymer. The typical methods are classified as follows:
(1) a. Crosslinking upon polymer-coating treatment of precursor particles,
   b. Crosslinking after polymer-coating treatment of precursor particles
(2) a. Addition of a crosslinking agent (including crosslinking reaction which proceeds at room temperature or low temperature),
   b. Introduction of a crosslinkable functional group into polymer
(3) a. Thermocrosslinking,
   b. Radiation crosslinking

It should be noted that the above methods (1), (2) and (3) can be used in combination. Examples of the combination of “(1) a”, “(2) a” and “(3) a” include a method wherein a heat treatment is performed with a bifunctional monomer upon providing a coating polymer by initiating polymerization from the surfaces of precursor particles or depositing the polymer on the surfaces of the precursor particles, and a method wherein a heat treatment is performed with a bifunctional monomer upon polymerizing by including the precursor particles in a monomer emulsion. With respect to the combination of “(1) b”, “(2) a” and “(3) a”, for example, a polyfunctional epoxy crosslinking agent is added and then a heating treatment for crosslinking is carried out after the coating polymer is provided by a deposition of a polymer having a carboxyl group or by a polymerization of a monomer having a carboxyl group. The same is true for the case wherein a hydroxyl group is used instead of the carboxyl group and an isocyanate crosslinking agent is used instead of the epoxy crosslinking agent. An example of “(2) b” includes a method wherein an epoxy group, an isocyanate group or a double bond is introduced into a coating polymer. In this case, “(3) a” can be used for the introduction of the epoxy group or isocyanate group, and also “(3) b” can be used for the introduction of the double bond.

In a case where a coating polymer is used, it should be understood that “substance to which a target substance can bind” or “functional group to which a target substance can bind” is immobilized on the body surface of the particles of the present invention and/or the surface of the coating polymer.

In a case where a coating polymer is provided on the surface of the particle body, there is no restriction on the method for immobilizing “functional group to which a target substance can bind”. That is to say, any suitable methods may be used as long as “functional group to which a target substance can bind” is allowed to attach or adhere to the particle body. Furthermore, “functional group to which a target substance can bind” may be immobilized prior to a provision of a coating polymer, during a provision of a coating polymer, or subsequent to a provision of a coating polymer.

In a case where a coating polymer is provided on the surface of the particle body, an example of the method for immobilizing “functional group to which a target substance can bind” includes a method wherein a monomer having “functional group to which a target substance can bind” is polymerized or copolymerized during a polymerization reaction of a polymer to be provided. Examples of the monomer having “functional group to which a target substance can bind” include (meth)acrylic acid, glycidyl (meth)acrylate, hydroxyalkyl (meth)acrylate, dimethylaminoalkyl (meth)acrylate, isocyanatoalkyl (meth)acrylate, p-styrenesulfonic acid (salt), dimethylolpropanoic acid, N-alkyldiethanolamine, (aminoethylamino)ethanol and lysine.

When “functional group having stronger binding properties to a target substance” is immobilized, and also a coating polymer is provided on the surface of the particle body, a compound may be additionally introduced into particles, the compound having two functional groups being “functional group b having reactivity with a functional group a introduced into the coating polymer by the above-described method” and “functional group c having higher binding properties to a target substance”. In this case, particles with “functional group c having higher binding properties to a target substance” immobilized thereon can be obtained by binding “functional group a” and “functional group b” to each other. When it is required to make a space between the surface of the coating polymer and “functional group to which a target substance can bind” or to make a space between the surface of the particle body and “functional group to which a target substance can bind” (namely, it is required to introduce a “linker”), a compound having two functional groups being “functional group b having reactivity with the introduced functional group a” and “functional group to which a target substance can bind” may be additionally introduced into particles with “functional group a” introduced thereto. Even in this case, “functional group to which a target substance can bind” is immobilized on particles via a bond between “functional group a” and “functional group b”. The linker may be more extended by repeating the introduction of the compound two or more times. When the space between the surface of the coating polymer and “functional group to which a target substance can bind” further increases, or the space between the surface of the particle body and “functional group to which a target substance can bind” further increases, it is expected to provide an advantageous effect. For example, the degree of freedom of “functional group to which a target substance can bind” increases and thus a reactivity is improved. In addition, the degree of freedom of the target substance increases and thus the function of the target substance is not inhibited. If the number of atoms existing from a backbone of the coating polymer to the functional group is defined as the length of a linker, the above advantageous effect can be expected when the length of the linker is in the range of 5 atoms to 50 atoms. It is particularly preferred that a biogenic-related substance having a low nonspecific adsorptivity (for example, a polyethylene glycol chain) is used as a backbone of the linker.

In a case where a coating polymer is provided on the surface of the particle body, there is no restriction on the method for immobilizing “substance to which a target substance can bind”. That is to say, any suitable methods may be used as long as “substance to which a target substance can bind” is allowed to attach or adhere to the particle body. As with the case of “functional group to which a target substance can bind”, “substance to which a target substance can bind” may be immobilized prior to a provision of a coating polymer, during a provision of a coating polymer, or subsequent to a provision of a coating polymer.

“Substance to which a target substance can bind” can be immobilized on the particles by the method similar to the above method for introducing “functional group to which a target substance can bind”. For example, a functional group having binding properties to “substance to which a target substance can bind” is preliminarily introduced onto the surface of the particle body or the surface of the coating polymer, and then “substance to which a target substance can bind” can be immobilized to the particles via the preliminarily introduced functional group. When not only the coating polymer but also “substance to which a target substance can bind” is hydrophobic, a so-called “hydrophobic interaction” can occur in water so that they are adsorbed with each other. In this way, the hydrophobic “substance to which a target substance can bind” can be immobilized on the surface of a coating polymer.

Hereinafter, the binding of the particles with the target substance will be described in detail. When the particles of the present invention and the target substance is are allowed to coexist, the target substance can bind the particles due to an adsorptivity or affinity generated between “substance or functional group capable of binding to a target substance” of the particle and the target substance. In the classification below, “adsorption” is defined to have the same meaning as “chemical adsorption”.

As an example of embodiment wherein a target substance binds to particles due to the adsorptivity, “target substance” is avidin, a particle body is made of zirconia, and “substance or functional group capable of binding to a target substance” is an epoxy group.

With respect to “affinity”, “substance or functional group capable of binding to a target substance” immobilized on the surface of the particle body can be roughly classified into the following five kinds, based on the kind of the affinity generated between “substance or functional group capable of binding to a target substance” and the target substance (it should be noted that substances or functional groups exemplified in each classification are only for illustrative purposes and other substances or functional groups are also included). When involved in the affinity as described above, “substance or functional group capable of bonding to a target substance” is hereinafter referred to also as “substance or functional group having affinity”.

(1) Examples of “substance or functional group having affinity with a target substance” wherein the affinity results from electrostatic interaction, π-π interaction, π-cation interaction, or dipole-dipole interaction:

silica, activated carbon, sulfonic acid group, carboxyl group, diethylaminoethyl group, triethylaminoethyl group, phenyl group, arginine, cellulose, lysin, polylysin, polyamide, poly(N-isopropylacrylamide), crown ether or cyclic compound having n electrons, and functional group derivatives, oxygen conjugates and fluorescence probe conjugates thereof.

(2) Examples of “substance or functional group having affinity with a target substance” wherein the affinity results from hydrophobic interaction:

alkyl group, octadecyl group, octyl group, cyanopropyl group, butyl group, phenyl group, and functional group derivatives, oxygen conjugates and fluorescence probe conjugates thereof.

(3) Examples of “substance or functional group having affinity with a target substance” wherein the affinity results from hydrogen bond:

DNA, RNA, Oligo (dT), chitin, chitosan, amylose, cellulose, dextrin, dextran, pullulan, polysaccharide, lysin, polylysin, polyamide, poly(N-isopropylacrylamide), β-glucan, and functional group derivatives, oxygen conjugates and fluorescence probe conjugates thereof.

(4) Examples of “substance or functional group having affinity with a target substance” wherein the affinity results from coordinate bond:

iminodiacetic acid, nickel, nickel ion, nickel complex, cobalt, cobalt ion, cobalt complex, copper, copper ion and copper complex, and oxygen conjugates and fluorescence probe conjugates thereof.

(5) Examples of “substance or functional group having affinity with a target substance” wherein the affinity results from a biochemical interaction (biochemical interaction means an interaction including an interaction relating to biological molecules, such as antigen-antibody reaction, ligand-receptor bond, hydrogen bond, coordinate bond, hydrophobic interaction, electrostatic interaction, π-π interaction, π-cation interaction, dipole-dipole interaction and van der Waals force acting alone or in combination thereof):

antigen, antibody, receptor, ligand, biotin, avidin, streptavidin, NeutrAvidin, silica, activated carbon, magnesium silicate, hydroxyapatite, albumin, amylose, cellulose, lectin, protein A, protein G, S protein, dextrin, dextran, pullulan, polysaccharide, calmodulin, nickel, nickel ion, nickel complex, cobalt, cobalt ion, cobalt complex, copper, copper ion, copper complex, gelatin, N-acetylglucosamine, iminodiacetic acid, aminophenylboric acid, ethylenediaminediacetic acid, aminobenzamidine, arginine, lysin, polylysin, polyamide, diethylaminoethyl group, triethylaminoethyl group, ECTEOLA-cellulose, fibronectin, vitronectin, peptides containing an arginine-glycine-aspartic (ROD) acid sequence, laminin, poly(N-isopropylacrylamide), collagen, concanavalin A, adenosine 5′phosphoric acid (ATP), ADP, ATP, nicotinamide adenine dinucleotide, acridine dye, aprotinin, ovomucoid, inhibitors (e.g. trypsin inhibitor and protease inhibitor), phosphorylethanolamine, phenylalanine, protamine, cibacron blue, Procion Red, heparin, glutathione, DIG, DIG antibody, DNA, RNA, Oligo (dT), chitin, chitosan, β-glucan, calcium phosphate, calcium hydrogenphosphate, hyaluronic acid, elastin, sericin and fibroin, and functional group derivatives, oxygen conjugates and fluorescence probe conjugates thereof.

As is apparent from the above classification, the expression “having affinity” as used herein substantially means that an electrostatic interaction, a π-π interaction, a π-cation interaction, a dipole-dipole interaction, a hydrophobic interaction, a biochemical interaction, a hydrogen bond or a coordinate bond is generated between a target substance and a substance or functional group immobilized on the particles. It should be noted that the substance or functional group may have two or more kinds of affinities according to the kind of the substance or functional group to be immobilized on the particle body and there may be overlapping substance or functional group in the above classification. There is no restriction on the above classification, and any suitable substances or functional groups may be immobilized on the particles as long as it has a function of acting on a target substance so as to allow the target substance to exist on the surfaces of particles or in the vicinity thereof. For example, substances or functional groups having affinity due to a complementary shape with a target substance may be immobilized.

There is no restriction on an embodiment or a method for immobilizing “substance or functional group having affinity with a target substance” on the surface of the precursor particles. For example, by providing a polymer, an inorganic compound, a low molecular weight linker or a coupling agent, “substance or functional group having affinity with a target substance” can be immobilized on the surface of the precursor particles due to a binding action, an adsorption action or an absorption action. Alternatively, “substance or functional group having affinity with a target substance” can be immobilized on the surface of the precursor particles by utilizing a conventional method for coating particles.

An example of the method for immobilizing “substance having affinity with a target substance” onto a particle body will be described. When an antibody is used as “substance having affinity with a target substance”, the above-described method for immobilizing a functional group by the use of siloxane can be employed. Specifically, a compound having a double bond and an epoxy group (e.g. glycidyl methacrylate) is reacted with the Si—H moiety of siloxane, and thereby the epoxy group is immobilized on the surfaces of particles. Subsequently, an aqueous mixture of the resultant particles and an antibody is stirred to produce the particles with the antibody immobilized thereon.

Even as a method for immobilizing “functional group having affinity with a target substance” onto the surfaces of particles, the above-described method using siloxane can be also employed, for example.

Hereinafter, a separation method using the particles of the present invention will be described in detail. This separation method is intended for separating a target substance from a sample by the use of the particles of the present invention, or intended for obtaining particles with a target substance immobilized thereon. The separation method of the present invention comprises the steps of:

(i) bringing particles) and a sample containing a target substances into contact with each other in order to bind the particle(s) and the target substance(s) to each other;

(ii) allowing the sample to stand in order to allow a spontaneous sedimentation of the particle(s) in the sample; and

(iii) recovering the particle(s) which has precipitated in the sample in order to separate the target substance(s) from the sample or obtain the particles) with the target substances) immobilized thereon.

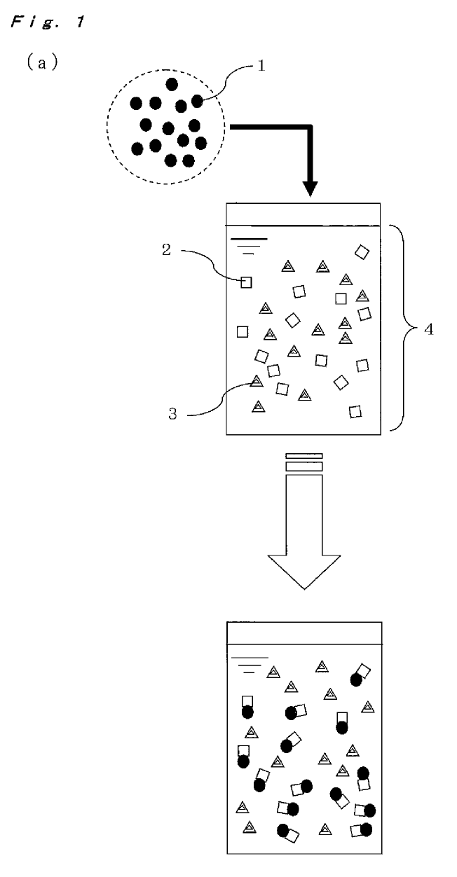

In the step (i), the particles of the present invention are brought into contact with the sample containing the target substance, and thereby the particles and the target substance are allowed to bind to each other (see FIG. 1(a)). In this regard, the sample and the particles are allowed to be in contact with each other by supplying the particles to the sample containing the target substance. If necessary, a stirring operation may be performed in order to promote the binding of the target substance to the particles. The particles to be supplied are not usually in a single form. That is to say, the particles may be supplied in powder form having an average size of 1 μm to 1 mm as described above. The amount of the particles in powder form varies depending on the kind of samples and separation applications. For example, only one particle may be used, but the amount of particles is usually up to in gram weight (i.e. from about 10⁻² g to 10³ g) for analytical and laboratory applications, whereas the amount of particles is from in kilogram weight (i.e. about 1 to 10³ kg) to in ton weight (i.e. about 1 to 10 ton) for industrial applications.

In order to ensure the spontaneous sedimentation of the particles in the step (ii), the sample containing the target substance is preferably used in a state of being filled in a beaker, a measuring cylinder, a test tube, a microtube, a biochip, a chemical chip or a μ-TAS chip.

The binding between the target substance and the particles is brought about by an adsorptive power or affinity acting between them. More specifically, the target substance and the particles can bind to each other by the action of an adsorptive power or affinity between the target substance and “substance or functional group capable of binding to the target substance” immobilized on the particle body. Depending on the amount of the particles to be supplied in powder form into the sample, there may exist particles which do not contribute to the binding of the target substance (particularly when an excessive amount of the particles are supplied). The particles used in the method of the present invention can suppress a nonspecific binding phenomenon in which “substances other than target substances” bind to the particles. Therefore, even when “substances other than target substances” are contained in the sample, the target substances can preferentially bind to the particles.

As described above, examples of the target substance include nucleic acids, proteins (e.g. avidin, biotinylated HRP and the like), sugars, lipids, peptides, cells, eumycetes (fungus), bacteria, yeasts, viruses, glycolipids, glycoproteins, complexes, inorganic substances, vectors, low molecular compounds, high molecular compounds, antibodies and antigens. As described above, examples of the sample include body fluids such as urine, blood, serum, plasma, sperm, saliva, sweat, tears, ascitic fluids and amniotic fluids from humans or animals; suspension liquids, extraction liquids, solutions and crushed solutions of organs, hair, skin, nail, bone, muscle and nervous tissue from humans or animals; suspension liquids, extraction liquids, solutions and crushed solutions of stools; suspension liquids, extraction liquids, solutions and crushed solutions of cultured cells or cultured tissues; suspension liquids, extraction liquids, solutions and crushed solutions of viruses; suspension liquids, extraction liquids, solutions and crushed solutions of fungus bodies; suspension liquids, extraction liquids, solutions and crushed solutions of soil; suspension liquids, extraction liquids, solutions and crushed solutions of plants; suspension liquids, extraction liquids, solutions and crushed solutions of food and processed food; and drainage water.

In the step (ii), the sample to which the particles have been supplied is allowed to stand in order for the particles of the present invention to spontaneously settle out in the sample (see FIG. 1(b)). Due to the fact that the particles used in the method of the present invention have the above-described density and specific surface area, a higher spontaneous sedimentation rate can be achieved. In other words, the particles of the present invention are not only high density particles, but also are regarded as substantially non-porous particles in terms of comparatively low specific surface of 0.0005 m²/g to 1.0 m²/g. This can prevent the particles from incorporating a gas (e.g. air) thereinto upon supplying the particles into a sample containing the target substances. Since a gas does not exist in the interior of the particle, any influence attributable to buoyancy of the gas can be excluded. As a result, a satisfactory separation rate can be achieved only by the spontaneous sedimentation of the particles.

In the step (iii), the particles which have precipitated in the sample are collected, and thereby the target substance is separated from the sample or the particles on which the target substance has been immobilized are obtained (see FIG. 1(c)). In this regard, the precipitated particles tend to aggregate in a lower region of the sample or a bottom region of a container due to spontaneous sedimentation, whereas a supernatant is formed in an upper region of the sample. Therefore, the precipitated particles can be recovered from the sample by withdrawing the supernatant by sucking using a pipette. Due to the fact that the target substance has bound to the recovered particles, the recovery of the particles can bring about a separation of the target substance from the sample.

In this way, according to the method of the present invention, the target substance can be separated or the particles with the target substance immobilized thereon can be obtained. By putting this method to practice use, analysis, extraction, purification and reaction of various target substances (e.g. cells, proteins, nucleic acids and chemical substances) can be realized. More specifically, the method of the present invention makes it possible to perform analysis, extraction, purification or reaction of target substances, in addition to the above separation or immobilization of target substances. For example, with respect to “method for analyzing a target substance”, the particles with “antibody capable of binding to substances to be detected” immobilized thereon are filled in a chip, and then “substances to be detected” is supplied to the chip so that “substances to be detected” hind to the particles. As a marker capable of binding to “substances to be detected”, an enzyme, a fluorescence dye (fluorocho) or a magnetic material is used by binding it to the antibody immobilized on each particle. Accordingly, the amount of “substances to be detected” is measured by light absorption, chemoluminescence, fluorescence or magnetism which arises from the marker. In this way, a quantitative analysis or qualitative analysis of “substances to be detected” can be performed. In a case where “substance to be detected” is a nucleic acid, the particles with “nucleic acid capable of binding to the nucleic acid to be detected” immobilized thereon are used. In this case, “nucleic acid to be detected” with an enzyme or a fluorescence dye attached thereto is supplied to a chip in which the particles is filled. As a result, “nucleic acid to be detected” is immobilized on the particles, and thereby the quantitative or qualitative analysis can be performed by light absorption, chemoluminescence, fluorescence or magnetism. In this regard, in each reaction stage, the reaction may be carried out in the same position or different positions of plural reaction vessels provided on the chip. Furthermore, for the purpose of performing a movement between plural reaction vessels provided on the chip, and also for the purpose of performing a stirring in each reaction vessel, a gravity is available. For “method for extracting a target substance” or “method for purifying a target substance”, subsequent to the separation of the step (iii), the target substance may be extracted or purified by the use of a substance capable of detaching or isolating the target substance from the particles, or by performing a required treatment such as heating or cooling. Furthermore, for “method for performing a reaction of a target substance”, a target substance is supplied to the chip wherein the particles with “substance capable of binding to the target substance” immobilized thereon are filled. As a result, the target substance is immobilized on the particles, and thereby the target substance is subject to the reaction by performing a mixing, heating, stirring or ultraviolet irradiation in each of plural reaction vessels provided on the chip. In this case, for the purpose of performing a movement between plural reaction vessels provided on the chip, and also for the purpose of performing a stirring in each reaction vessel, a gravity is available. It is also possible that an enzyme or catalyst is immobilized on the particles and subsequently they are supplied into a reaction system by the force of gravity.

Although a few embodiments of the present invention have been hereinbefore described, the present invention is not limited to these embodiments. It will be readily appreciated by those skilled in the art that various modifications are possible without departing from the scope of the present invention.

For example, (1) in order to suppress a nonspecific binding or nonspecific adsorption to particles upon separation of a target substance; (2) in order to control affinity of the particles; or (3) in order to use as a base material for introducing a functional group, at least one kind of a substance selected from the group consisting of polyethylene glycol, polyvinyl alcohol, polyvinyl pyrrolidone, polyacrylic acid, poly(2-ethyl-2-oxazoline), polydimethylacrylamide, dextran, pullulan, agarose, sepharose, amylose, cellobiose, chitin, chitosan, polysaccharide, normal serum, bovine serum albumin, human serum albumin, casein, skimmilk powder and functional group derivatives thereof may be adhered on the surface of the particle body. The method for adhering the above substance is not limited, and any suitable conventional methods for coating particles may be used. In this case, in a case where polyethylene glycol is used for example, the immobilized “substance or functional group capable of binding to a target substance” and the polyethylene glycol are present on the surface of each particle body.

The present invention as described above includes the following aspects:

First aspect: A particle to which a target substance can bind, characterized in that:

“substance or functional group capable of binding to the target substance” is immobilized on a surface of a particle body;

a density of the particle is in the range of 3.5 g/cm³ to 9.0 g/cm³; and

the particle body has no through-pore.

Second aspect: The particle according to the first aspect, characterized in that a specific surface area of the particle is in the range of 0.0005 m²/g to 1.0 m²/g.

Third aspect: The particle according to the first or the second aspect, characterized in that a coating of polymer is provided on a part of the surface of the particle body; and

“substance or functional group capable of binding to the target substance” is immobilized on the surface of the particle body or a surface of the polymer.

Fourth aspect: The particle according to the third aspect, characterized in that the coating of polymer is provided over an entire surface of the particle body; and

“substance or functional group capable of binding to the target substance” is immobilized on the surface of the polymer.

Fifth aspect: The particle according to the third or the fourth aspect, characterized in that the polymer is at least one kind of a polymer selected from the group consisting of polystyrene, poly(meth)acrylic acid, poly(meth)acrylic acid ester, polyvinylether, polyurethane, polyamide, polyvinyl acetate, polyvinyl alcohol, polyallylamine and polyethyleneimine.

Sixth aspect: The particle according to any one of the third to the fifth aspects, characterized in that the polymer is a crosslinked polymer.

Seventh aspect: The particle according to any one of the first to the sixth aspects, characterized in that the particle is a non-porous particle.

Eighth aspect: The particle according to any one of the first to the seventh aspects, characterized in that the particle body is made of at least one kind of a material selected from the group consisting of zirconia (e.g. zirconium oxide, yttrium-added zirconium oxide), iron oxide and alumina.

Ninth aspect: The particle according to any one of the first to the eighth aspects, characterized in that the particle exhibits magnetism.

Tenth aspect: The particle according to the ninth aspect, characterized in that a saturation magnetization is in the range of 0.5 to 85 A·m²/kg.

Eleventh aspect: The particle according to any one of the first to the tenth aspects, characterized in that an average size of the particle is in the range of 1 μm to 1 mm.

Twelfth aspect: The particle according to any one of the first to the eleventh aspects, characterized in that “substance capable of binding to the target substance” is at least one kind of a substance selected from the group consisting of biotin, avidin, streptavidin and neutravidin.

Thirteenth aspect: The particle according to any one of the first to the eleventh aspects, characterized in that “functional group capable of binding to the target substance” is at least one kind of a functional group selected from the group consisting of carboxyl group, hydroxyl group, epoxy group, tosyl group, succinimide group, maleimide group, thiol group, thioether group, disulfide group, aldehyde group, azido group, hydrazide group, primary amino group, secondary amino group, tertiary amino group, imide ester group, carbodiimide group, isocyanate group, iodoacetyl group, halogen-substitution product of carboxyl group and double bond.

Fourteenth aspect: The particle according to any one of the third to the thirteenth aspects, characterized in a silicon-containing substance and/or polyethylene glycol is present on at least a part of the surface of the particle body and/or the surface of the polymer.

Fifteenth aspect: The particle according to any one of the first to the fourteenth aspects, characterized in that the target substance can bind to the particle by an adsorbability or affinity generated between the target substance and “substance or functional group capable of binding to the target substance”.

Sixteenth aspect: The particle according to the fifteenth aspect, characterized in that the affinity generated between the target substance and “substance or functional group capable of binding to the target substance” is due to an electrostatic interaction, π-π interaction, π-cation interaction, dipole-dipole interaction, hydrophobic interaction, hydrogen bond, coordinate bond or biochemical interaction.

Seventeenth aspect: A method for separating a target substance from a sample or obtaining a particle with a target substance immobilized thereon, by using of the particle according to any one of the first to the sixteenth aspects, the method comprising the steps of:

(i) bringing the particle and the sample containing the target substance into contact with each other, and thereby binding the particle and the target substance to each other,

(ii) allowing the sample to stand, and thereby allowing a natural sedimentation of the particle in the sample, and

(iii) recovering the particle precipitated in the sample, and thereby separating the target substance from the sample or obtaining the particle with the target substance immobilized thereon.

Eighteenth aspect: A method for performing an analysis, extraction, purification or reaction of a target substance by utilization of the method according to the seventeenth aspect.

### INDUSTRIAL APPLICABILITY

The particles of the present invention can be used for a quantitative determination, separation, purification, analysis and the like of target substances such as cells, proteins, nucleic acids and chemical substances. For example, the particles of the present invention capable of binding to nucleic acids such as DNA can be used for analysis of DNA, and thus they contribute to tailor-made medical technologies.

### EXAMPLES

The following Examples and Comparative Examples were carried out so as to confirm a separation rate and nonspecific binding characteristics of particles.

Preparation of Particles

In Examples 1 to 16 and Comparative Examples 1 to 9, particles were prepared in the following manner.

### Example 1

Particles prepared in Example 1 are yttrium-doped zirconia particles P₁ with a hydroxyl group immobilized thereon. First, yttrium-doped zirconia particles p₁ manufactured by NIKKATO CORPORATION were prepared. The particles p₁ had a particle size of 50 μm, a specific surface area of 0.02 m²/g and a density of 6 g/cm³. 1 g of the particles p₁ were dispersed into toluene and 0.5 g of 1,3,5,7-tetramethylcyclotetrasiloxane (manufactured by Shin-Etsu Chemical Co., Ltd., LS-8600) was added to the resultant dispersion. The dispersion was evaporated to remove toluene, and then the particles were allowed to stand in a vacuum desiccator at 50° C. for 4 hours. Subsequently, the particles p₁ were heated in a thermostatic bath at 150° C. for 1.5 hours. It was confirmed that such a treatment made the particles p₁ hydrophobic and thus a coating of 1,3,5,7-tetramethylcyclotetrasiloxane was formed on the particles p₁.

Subsequently, the resultant particles were dispersed in water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. Subsequently, the particles were washed, filtered and then dried to obtain yttrium-doped zirconia particles P₁ with a hydroxyl group immobilized on the surface thereof. The particles P₁ were hydrophilic particles. The particles P₁ had a specific surface area of 0.02 m²/g, a density of 6 g/cm³ and a particle size of about 50 μm (a difference between the particle size of the particles P₁ and the particle size of the zirconia particles p₁ is within a measurement deviation).

### Example 2

Particles prepared in Example 2 are yttrium-doped zirconia particles P₂ with a hydroxyl group immobilized thereon. Particles P₂ are different from the particles P₁ of Example 1 in that the particles P₂ is magnetic particles.

First, yttrium-doped zirconia particles p₂ manufactured by NIKKATO CORPORATION were prepared. The particles p₂ had a particle size of 50 μm, a specific surface area of 0.02 m²/g and a density of 6 g/cm³. 1 g of particles p₂ were dispersed in water and a silane coupling agent (manufactured by Shin-Etsu Chemical Co., Ltd., KBM-903) was added to the resultant dispersion, and thereby allowing the silane coupling agent to deposit on the surface of the particles p₂. Then, a Pd catalyst Catalyst-6F manufactured by SHIPLEY FAR EAST LTD. was added into the dispersion to form plating nuclei on the surface of the particles p₂. The resultant particles was washed with 1.2N hydrochloric acid and a magnetic nickel plating layer was formed on the surface of the particles using a nickel plating solution Topnicolon LPH manufactured by OKUNO CHEMICAL INDUSTRIES CO., LTD. The particles were washed, filtered and then dried. In the subsequent process, the same treatment as in Example 1 was carried out to obtain particles P₂ with a hydroxyl group immobilized thereon. Namely, the resultant particles were dispersed in water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. The particles were washed, filtered and then dried to obtain yttrium-doped zirconia particles P₂ with a hydroxyl group immobilized on the surface thereof. The particles P₂ were hydrophilic particles. The particles P₂ had a specific surface area of 0.02 m²/g, a density of 6 g/cm³ and a particle size of about 50 μm (a difference between the particle size of the particles P₂ and the particle size of the zirconia particles p₂ is within a measurement deviation). The amount of saturation magnetization of the particles P₂ was measured. As a result, it was 4.5 A·m²/kg.

### Example 3

Particles prepared in Example 3 are yttrium-doped zirconia particles P₃ with a hydroxyl group immobilized thereon. Particles P₃ are different from the particles of Example 1 in terms of a method for preparation of particles.

First, yttrium-doped zirconia particles p₃ manufactured by NIKKATO CORPORATION were prepared. The particles p₃ had a particle size of 50 μm, a specific surface area of 0.02 m²/g and a density of 6 g/cm³. 10 g of particles p₃ was dispersed in 25 g of pure water and then 3 g of 3-glycidoxypropyltrimethoxysilane having an epoxy group at the end was added to the resultant dispersion, followed by stirring for 4 hours. After washing the particles with water, a dispersion obtained by supplying 10 ml of 10 wt % ethanolamine to the particles was stirred at room temperature for 12 hours. The particles were washed, filtered and then dried to obtain yttrium-doped zirconia particles P₃ with a hydroxyl group immobilized on the surface. The particles P₃ were hydrophilic particles. The particles P₃ had a specific surface area of 0.02 m²/g, a density of 6 g/cm³ and a particle size of about 50 μm (a difference between the particle size of the particles P₃ and the particle size of the zirconia particles p₃ is within a measurement deviation).

### Example 4

Particles prepared in Example 4 are yttrium-doped zirconia particles P₄ with a hydroxyl group immobilized thereon. The method for preparation of particles P₄ differs from those of Examples 1 and 3.

First, yttrium-doped zirconia particles p₄ manufactured by NIKKATO CORPORATION were prepared. The particles p₄ had a particle size of 50 μm, a specific surface area of 0.02 m²/g and a density of 6 g/cm³. 10 g of particles p₄ were dispersed into 25 g of pure water, and then 5 g of tetraethoxysilane and 5 g of ammonia water were added to the resultant dispersion, followed by stirring for 4 hours. Subsequently, 3 g of 3-glycidoxypropyltrimethoxysilane having an epoxy group at the end was added to the dispersion, followed by stirring for 3 hours. After washing the particles with water, a dispersion obtained by supplying 10 ml of 10 wt % ethanolamine to the particles was stirred at room temperature for 12 hours. The particles were washed, filtered and then dried to obtain yttrium-doped zirconia particles P₄ with a hydroxyl group immobilized on the surface. The particles P₄ were hydrophilic particles. The particles P₄ had a specific surface area of 0.02 m²/g, a density of 6 g/cm³ and a particle size of about 50 μm (a difference between the particle size of the particles P₄ and the particle size of the zirconia particles p₄ is within a measurement deviation).

### Example 5

Particles prepared in Example 5 are yttrium-doped zirconia particles P₅ with avidin immobilized thereon.

First, yttrium-doped zirconia particles p₅ manufactured by NIKKATO CORPORATION were prepared. The particles p₅ had a particle size of 50 μm, a specific surface area of 0.02 m²/g and a density of 6 g/cm³. 10 g of particles p₅ were dispersed into 25 g of pure water and then 3 g of 3-glycidoxypropyltrimethoxysilane was added into the resultant dispersion while stirring, followed by stirring for 4 hours. After washing particles with acetone, the particles were vacuum-dried to obtain yttrium-doped zirconia particles having an epoxy group.

Subsequently, an aqueous solution prepared by dissolving 100 mg of avidin in 20 ml of 10 mM PBS solution (pH 7.2) was added to 200 mg of the resultant particles, followed by stirring overnight. The particles were washed with a 10 mM PBS solution (pH 7.2) and water and then vacuum-dried to obtain yttrium-doped zirconia particles P₅ with avidin immobilized thereon. The resultant particles P₅ had a specific surface area of 0.02 m²/g, a density of 6 g/cm³ and a particle size of about 50 μm (a difference between the particle size of the particles P₅ and the particle size of the zirconia particles p₅ is within a measurement deviation).

### Example 6

Particles prepared in Example 6 are alumina particles P₆ with avidin immobilized thereon. The alumina particles P₆ are different from the particles of Example 5 in terms of the precursor particles and the preparation method.

First, alumina particles p₆ manufactured by TAIMEI Chemicals Co., Ltd were prepared. The particles p₆ had a particle size of 200 μm, a specific surface area of 0.008 m²/g and a density of 3.6 g/cm³. 1 g of particles p₆ were dispersed into toluene and then 0.5 g of 1,3,5,7-tetramethylcyclotetrasiloxane (manufactured by Shin-Etsu Chemical Co., Ltd., LS-8600) was added to the resultant dispersion. The dispersion was evaporated to remove toluene and the particles were allowed to stand in a vacuum desiccator at 50° C. for 4 hours. Subsequently, the particles p₆ were heated in a thermostatic bath at 150° C. for 1.5 hours. It was confirmed that such a treatment made the particles p₆ hydrophobic and thus a coating of 1,3,5,7-tetramethylcyclotetrasiloxane was formed on the particles p₆.

Subsequently, the resultant particles were dissolved into water and then heated up to 80° C. 10 mg of chloroplatinic acid and 0.5 g of LIGHT-ESTER manufactured by KYOEISHA CHEMICAL Co., LTD. were added to the resultant, followed by stirring at 80° C. for 4 hours. After washing the particles with water, a dispersion obtained by supplying 10 ml of 10 wt % ethanolamine to the particles was stirred at room temperature for 12 hours. Subsequently, the particles were washed, filtered and then dried to obtain alumina particles with a hydroxyl group immobilized on the surface thereof. The particles were hydrophilic particles.

Subsequently, an aqueous solution prepared by dissolving 100 mg of avidin in 20 ml of a 10 mM PBS solution (pH 7.2) was added to 200 mg of the resultant particles, followed by stirring overnight. After washing the particles with a 10 mM PBS solution (pH 7.2) and water, the particles were vacuum-dried to obtain alumina particles P₆ with avidin immobilized thereon. The resultant particles P₆ had a specific surface area of 0.008 m²/g, a density of 3.6 g/cm³ and a particle size of about 200 μm (a difference between the particle size of the particles P₆ and the particle size of the zirconia particles p₆ is within a measurement deviation).

### Example 7

Particles prepared in Example 7 are copper particles P₇ with avidin immobilized thereon. The copper particles P₇ are different from the particles of Example 6 in terms of the precursor particles.

First, copper particles p₇ manufactured by Hitachi Metals, Ltd. were prepared. The particles p₇ had a particle size of 50 μm, a specific surface area of 0.013 m²/g and a density of 8.9 g/cm³. 1 g of particles p₇ was dispersed into toluene and then 0.5 g of 1,3,5,7-tetramethylcyclotetrasiloxane (manufactured by Shin-Etsu Chemical Co., Ltd., LS-8600) was added to the resultant dispersion. The dispersion was evaporated to remove toluene, and then the particles were allowed to stand in a vacuum desiccator at 50° C. for 4 hours. Subsequently, the particles p₇ were heated in a thermostatic bath at 150° C. for 1.5 hours. It was confirmed that such a treatment made the particles p₇ hydrophobic and thus a coating of 1,3,5,7-tetramethylcyclotetrasiloxane was formed on the particles p₇.

Subsequently, the resultant particles were dispersed into water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours, After washing the particles with water, a dispersion obtained by supplying 10 ml of 10 wt % ethanolamine to the particles was stirred at room temperature for 12 hours. Subsequently, the particles were washed, filtered and then dried to obtain copper particles with a hydroxyl group immobilized on the surface thereof. The resultant particles were hydrophilic particles.

Subsequently, an aqueous solution prepared by dissolving 100 mg of avidin in 20 ml of a 10 mM PBS solution (pH 7.2) was added to 200 mg of the resultant particles, followed by stirring overnight. After washing the particles with a 10 mM PBS solution (pH 7.2) and water, the particles were vacuum-dried to obtain copper particles P₇ with avidin immobilized thereon. The resultant particles P₇ had a specific surface area of 0.013 m²/g, a density of 8.9 g/cm³ and a particle size of about 50 μm (a difference between the particle size of the particles P₇ and the particle size of the zirconia particles p₇ is within a measurement deviation).

### Example 8

Particles prepared in Example 8 are yttrium-doped zirconia particles P₈ with avidin immobilized thereon. The yttrium-doped zirconia particles P₈ and the particles of Example 6 and Example 7 differ in terms of the precursor particle. The yttrium-doped zirconia particles P₈ are identical to the particles of Example 5 in that the precursor particles are made of yttrium-doped zirconia. However, the precursor particles of Example 8 are made of yttrium-doped zirconia having physical properties which are different from that of Example 5.

First, yttrium-doped zirconia particles p₈ manufactured by NIKKATO CORPORATION were prepared. The particles p₈ had a particle size of 30 μm, a specific surface area of 0.03 m²/g and a density of 6 g/cm³. 1 g of particles p₈ were dispersed into toluene and then 0.5 g of 1,3,5,7-tetramethylcyclotetrasiloxane (manufactured by Shin-Etsu Chemical Co., Ltd., LS-8600) were added to the resultant dispersion. The dispersion was evaporated to remove toluene and then the particles were allowed to stand in a vacuum desiccator at 50° C. for 4 hours. Subsequently, the particles p₈ were heated in a thermostatic bath at 150° C. for 1.5 hours. It was confirmed that such a treatment made the particles p₈ hydrophobic and thus a coating of 1,3,5,7-tetramethylcyclotetrasiloxane was formed on the particles p₈.

Subsequently, the resultant particles were dispersed in water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. Subsequently, the particles were washed, filtered and then dried to obtain yttrium-doped zirconia particles with a hydroxyl group immobilized on the surface thereof. The resultant particles were hydrophilic particles.

Subsequently, an aqueous solution prepared by dissolving 100 mg of avidin in 20 ml of a 10 mM PBS solution (pH 7.2) was added to 200 mg of the resultant particles, followed by stirring overnight. After washing the particles with a 10 mM PBS solution (pH 7.2) and water, the particles were vacuum-dried to obtain yttrium-doped zirconia particles P₈ with avidin immobilized thereon. The resultant particles P₈ had a specific surface area of 0.03 m²/g, a density of 6 g/cm³ and a particle size of about 30 μm (a difference between the particle size of the particles P₈ and the particle size of the zirconia particles p₈ is within a measurement deviation).

### Example 9

Particles prepared in Example 9 are yttrium-doped zirconia particles P₉ with avidin immobilized thereon. The yttrium-doped zirconia particles P₉ are different from the particles of Example 5 and Example 8 in terms of the precursor particles. The yttrium-doped zirconia particles P₉ are identical to the particles of Example 5 in that the precursor particles are made of yttrium-doped zirconia. However the precursor particles of Example 9 are made of yttrium-doped zirconia having physical properties which are different from those of Examples 5 and 8.

First, yttrium-doped zirconia particles p₉ manufactured by Neturen Co., Ltd. were prepared. The particles p₉ had a particle size of 15 μm, a specific surface area of 0.04 m²/g and a density of 6 g/cm³. 1 g of particles p₉ were dispersed into toluene and 0.5 g of 1,3,5,7-tetramethylcyclotetrasiloxane (manufactured by Shin-Etsu Chemical Co., Ltd., LS-8600) were added to the resultant dispersion. The dispersion was evaporated to remove toluene and then the particles were allowed to stand in a vacuum desiccator at 50° C. for 4 hours. Subsequently, the particles p₉ were heated in a thermostatic bath at 150° C. for 1.5 hours. It was confirmed that such a treatment made the particles p₉ hydrophobic and a coating of 1,3,5,7-tetramethylcyclotetrasiloxane was formed on the particles p₉.

Subsequently, the resultant particles were dispersed in water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. Subsequently, the particles were washed, filtered and then dried to obtain yttrium-doped zirconia particles P₉ with a hydroxyl group immobilized on the surface thereof. The resultant particles were hydrophilic particles.

Subsequently, an aqueous solution prepared by dissolving 100 mg of avidin in 20 ml of a 10 mM PBS solution (pH 7.2) was added to 200 mg of the resultant particles, followed by stirring overnight. After washing the particles with a 10 mM PBS solution (pH 7.2) and water, the particles were vacuum-dried to obtain yttrium-doped zirconia particles P₉ with avidin immobilized thereon. The resultant particles P₉ had a specific surface area of 0.04 m²/g, a density of 6 g/cm³ and a particle size of about 15 μm (a difference between the particle size of the particles P₉ and the particle size of the zirconia particles p₉ is within a measurement deviation).

### Example 10

Particles prepared in Example 10 are polystyrene-coated zirconia particles P₁₀ with an epoxy group immobilized thereon. The particles have a feature that a coating of polymer is formed on at least a part of a particle body.

First, yttrium-doped zirconia particles p₁₀ manufactured by NIKKATO CORPORATION were prepared. The zirconia particles p₁₀ had a particle size of 30 μm, a specific surface area of 0.03 m²/g and a density of 6 g/cm³. 3 g of the zirconia particles p₁₀ were dispersed into a water-alcohol mixed solution and 0.13 g of methacryloxypropyltrimethoxysilane was added thereto, followed by stirring at 35° C. for about 30 minutes. After bubbling of nitrogen for 30 minutes, 0.1 g of sodium p-styrene sulfonate, 0.1 g of potassium persulfate, 9.4 g of a styrene monomer and 1.4 g of glycidyl methacrylate were added, followed by subjecting to the reaction at 70° C. for 8 hours. After the completion of the reaction, unreacted material and particles sedimenting slowly were removed by washing with water to obtain polystyrene-coated zirconia particles P₁₀ with an epoxy group immobilized thereon. The resultant particles P₁₀ had a specific surface area of 0.05 m²/g, a density of 5.5 g/cm³ and a particle size of about 30 μm (a difference between the particle size of the particles P₁₀ and the particle size of the zirconia particles p₁₀ is within a measurement deviation). The specific surface area of the resultant particles P₁₀ increases when compared with that of the precursor particles p₁₀. It is presumed that one of the reasons is that a portion of a polymer is deposited in a granular form.

### Example 11

Particles prepared in Example 11 are polystyrene-coated zirconia particles P₁₁ with avidin immobilized thereon. The polystyrene-coated zirconia particles P₁₁ are different from the particles P₁₀ of Example 10 in that the avidin is immobilized instead of an epoxy group.

200 mg of the “polystyrene-coated zirconia particles P₁₀ with an epoxy group immobilized thereon” obtained in Example 10 were supplied to an aqueous solution prepared by dissolving 100 mg of avidin in 20 ml of a 10 mM PBS solution (pH 7.2), followed by stirring overnight. After washing the particles with a 10 mM PBS solution (pH 7.2) and water, the particles were vacuum-dried to obtain polystyrene-coated zirconia particles P₁₁ with avidin immobilized thereon. The resultant particles P₁₁ had a specific surface area of 0.05 m²/g, a density of 5.5 g/cm³ and a particle size of about 30 μm (a difference between the particle size of the particles P₁₁ and the particle size of the zirconia particles p₁₁ is within a measurement deviation). The specific surface area of the resultant particles P₁₁ increases when compared with that of the precursor particles p₁₀. As with Example 10, it is presumed that one of the reasons is that a portion of a polymer is deposited in a granular form.

### Example 12

Particles prepared in Example 12 are crosslinked polystyrene-coated zirconia particles P₁₂ with an epoxy group immobilized thereon. The crosslinked polystyrene-coated zirconia particles P₁₂ are different from the particles P₁₀ of Example 10 in that polystyrene of a coating polymer is crosslinked.

The same treatment as in the case of Example 10 was carried out except that 0.3 g of divinylbenzene was used as a crosslinking agent for the styrene monomer of Example 10. As a result, “crosslinked polystyrene-coated zirconia particles P₁₂ with an epoxy group immobilized thereon” was obtained. The resultant particles P₁₂ had a specific surface area of 0.05 m²/g, a density of 5.5 g/cm³ and a particle size of about 30 μm (a difference between the particle size of the particles P₁₂ and the particle size of the zirconia particles p₁₀ is within a measurement deviation). The specific surface area of the resultant particles P₁₂ increases when compared with the precursor particles p₁₀. As with Example 1, it is presumed that one of the reasons is that a portion of a polymer is deposited in a granular form.

### Example 13

Particles prepared in Example 13 are polystyrene-coated magnetic zirconia particles P₁₃ with an epoxy group immobilized thereon. The polystyrene-coated magnetic zirconia particles P₁₃ are different from the particles of Example 10 in that the particles P₁₃ are magnetic particles.

First, yttrium-doped zirconia particles p₁₃ manufactured by NIKKATO CORPORATION were prepared. The particles p₁₃ had a particle size of 30 μm, a specific surface area of 0.03 m²/g and a density of 6 g/cm³. 1 g of particles p₁₃ were dispersed into water and a silane coupling agent (manufactured by Shin-Etsu Chemical Co., Ltd., KBM-903) was added to the resultant dispersion, and thereby allowing a silane coupling agent to deposit on the surface of the particles p₁₃. Then, a Pd catalyst Catalyst-6F manufactured by SHIPLEY FAR EAST LTD. was added to produce plating nuclei on the surface of the particles p₁₃. The resultant particles were washed with 1.2N hydrochloric acid and a nickel plating layer was formed on the surface of the particles using a nickel plating solution Topnicolon LPH manufactured by OKUNO CHEMICAL INDUSTRIES CO., LTD. The particles were washed, filtered and then dried. In the subsequent process, the same treatment as in Example 10 was carried out to obtain “polystyrene-coated magnetic zirconia particles P₁₃ with an epoxy group immobilized thereon”. The resultant particles P₁₃ had a specific surface area of 0.05 m²/g, a density of 6.5 g/cm³ and a particle size of about 32 μm. The amount of saturation magnetization of the particles P₁₃ was measured. As a result, it was 6.5 A·m²/kg. The specific surface area of the resultant particles P₁₃ increases when compared with that of the precursor particles p₁₀. As with Example 10, it is presumed that one of the reasons is that a portion of a polymer is deposited in a granular form.

### Example 14

Particles prepared in Example 14 are yttrium-doped zirconia particles P₁₄ with an “anti-human CRP monoclonal antibody 6404” immobilized thereon. First, yttrium-doped zirconia particles p₁₄ manufactured by NIKKATO CORPORATION were prepared. The particles p₁₄ had a particle size of 50 μm, a specific surface area of 0.02 m²/g and a density of 6 g/cm³. 1 g of the particles p₁₄ were dispersed into toluene and 0.5 g of 1,3,5,7-tetramethylcyclotetrasiloxane (manufactured by Shin-Etsu Chemical Co., Ltd., LS-8600) was added to the resultant dispersion. The dispersion was evaporated to remove toluene, and then the particles were allowed to stand in a vacuum desiccator at 50° C. for 4 hours. Subsequently, the particles p₁₄ were heated in a thermostatic bath at 150° C. for 1.5 hours. It was confirmed that such a treatment made the particles p₁₄ hydrophobic and a coating of 1,3,5,7-tetramethylcyclotetrasiloxane was formed on the particles p₁₄.

Subsequently, the resultant particles were dispersed into water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. The particles were washed, filtered and then dried to obtain yttrium-doped zirconia particles P₁₄′ with a hydroxyl group immobilized on the surface thereof. The particles P₁₄′ were hydrophilic particles.

Tosyl chloride was added to the particles P₁₄′, followed by stirring. The resultant particles were washed to obtain tosyl group activated zirconia particles. On the tosyl group activated zirconia particles, an anti-human CRP monoclonal antibody 6404 (manufactured by MedixBiochemica) was immobilized. It was confirmed by color development using a HRP-Rabbit-Anti-Mouse IgG2a secondary antibody (manufactured by ZYMED) that an anti-human CRP monoclonal antibody 6404 is immobilized on the surface of the particles. The resultant particles P₁₄ had a specific surface area of 0.02 m²/g, a density of 6 g/cm³ and a particle size of 50 μm.

### Example 15

Particles prepared in Example 15 are yttrium-doped zirconia particles P₁₅ with a hydroxyl group immobilized thereon. The yttrium-doped zirconia particles P₁₅ are different from the particles of Example 1 in terms of the method for preparation of particles.

First, yttrium-doped zirconia particles p₁₅ manufactured by NIKKATO CORPORATION were prepared. The particles p₁₅ had a particle size of 50 μm, a specific surface area of 0.02 m²/g and a density of 6 g/cm³. 1 g of particles p₁₅ was dispersed into water. A solution prepared by mixing KBE-402 (manufactured by Shin-Etsu Chemical Co., Ltd.) with ethanol was added in a form of drops to the resultant dispersion. The ammonia water is added to the dispersion, followed by stirring at room temperature for 4 hours. After washing the particles with water, a dispersion obtained by supplying 10 ml of 10 wt % ethanolamine to the particles was stirred at room temperature for 12 hours. Subsequently, the particles were washed, filtered and then dried to obtain yttrium-doped zirconia particles P₁₅ with a hydroxyl group immobilized on the surface thereof. The particles P₁₅ were hydrophilic particles. The particles P₁₅ had a specific surface area of 0.02 m²/g, a density of 6 g/cm³ and a particle size of about 50 μm (a difference between the particle size of the particles P₁₅ and the particle size of the zirconia particles p₁₅ is within a measurement deviation).

### Example 16

Particles prepared in Example 16 are yttrium-doped zirconia particles P₁₆ with avidin immobilized on the particles of Example 1.

An aqueous solution prepared by dissolving 100 mg of avidin in 20 ml of a 10 mM PBS solution (pH 7.2) was added to 200 mg of the particles obtained in Example 1, followed by stirring overnight. After washing the particles with a 10 mM PBS solution (pH 7.2) and water, the particles were vacuum-dried to obtain yttrium-doped zirconia particles P₁₆ with avidin immobilized thereon. The resultant particles P₁₆ had a specific surface area of 0.02 m²/g, a density of 6 g/cm³ and a particle size of about 50 μm.

Although the above Examples include an example using a silane coupling agent having an epoxy group, it should be noted that a silane coupling agent having a mercapto group or a functional group having a double bond may also be used instead.

### Comparative Example 1

Particles prepared in Comparative Example 1 are crosslinked acrylic particles R₁ with a hydroxyl group immobilized thereon.

First, crosslinked acrylic particles r₁ manufactured by Soken Chemical & Engineering Co., Ltd were prepared. The particles r₁ had a particle size of 30 μm, a specific surface area of 0.033 m²/g and a density of 1.19 g/cm³. 1 g of the particles r₁ were dispersed into toluene and 1 g of 1,3,5,7-tetramethylcyclotetrasiloxane (manufactured by Shin-Etsu Chemical Co., Ltd., LS-8600) was added to the resultant dispersion. The dispersion was evaporated to remove toluene, and then the particles were allowed to stand in a vacuum desiccator at 50° C. for 4 hours. Subsequently, the particles were heated in a thermostatic bath at 150° C. for 1.5 hours. It was confirmed that such a treatment made the particles r₁ hydrophobic and a coating of 1,3,5,7-tetramethylcyclotetrasiloxane was formed on the particles r₁.

Subsequently, the resultant particles were dispersed in water and then heated up to 80° C. 20 mg of chloroplatinic acid and 1 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 20 ml of 10 wt % ethanolamine to the particles was stirred at room temperature for 12 hours. The particles were washed, filtered and then dried to obtain crosslinked acrylic particles R₁ with a hydroxyl group immobilized on the surface thereof. The particles R₁ were hydrophilic particles. The particles R₁ had a specific surface area of 0.033 m²/g, a density of 1.19 g/cm³ and a particle size of about 30 μm (a difference between the particle size of the particles R₁ and the particle size of the zirconia particles r₁ is within a measurement deviation).

### Comparative Example 2

Particles prepared in Comparative Example 2 are porous zeolite particles R₂ with avidin immobilized thereon.

First, zeolite particles (HSZ-700) r₂ manufactured by TOSOH Corporation were prepared. The particles r₂ had a particle size of 18 μm, a specific surface area of 170 m²/g and a density of 2.3 g/cm³. 1 g of particles r₂ were dispersed into toluene and 2 g of 1,3,5,7-tetramethylcyclotetrasiloxane (manufactured by Shin-Etsu Chemical Co., Ltd., LS-8600) were added to the resultant dispersion. The dispersion was evaporated to remove toluene, and then the particles were allowed to stand in a vacuum desiccator at 50° C. for 4 hours. Subsequently, the particles were heated in a thermostatic bath at 150° C. for 1.5 hours. It was confirmed that such a treatment made the particles r₂ hydrophobic and a coating of 1,3,5,7-tetramethylcyclotetrasiloxane was formed on the particles.

Subsequently, the resultant particles were dispersed into water and then heated up to 80° C. 20 mg of chloroplatinic acid and 2 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 20 ml of 10 wt % ethanolamine to the particles was stirred at room temperature for 12 hours. The particles were washed, filtered and then dried to obtain porous zeolite particles r₂ with a hydroxyl group immobilized on the surface thereof. The particles r₂ were hydrophilic particles.

Subsequently, an aqueous solution prepared by dissolving 100 mg of avidin in 20 ml of 10 mM PBS solution (pH 7.2) was supplied to 200 mg of the resultant particles, followed by stirring overnight. The particles were washed with a 10 mM PBS solution (pH 7.2) and water and then vacuum-dried to obtain porous zeolite particles R₂ with avidin immobilized thereon. The resultant particles R₂ had a specific surface area of 170 m²/g, a density of 2.3 g/cm³ and a particle size of about 18 μm (a difference between the particle size of the particles R₂ and the particle size of the zirconia particles r₂ is within a measurement deviation).

### Comparative Example 3

Particles prepared in Comparative Example 3 are silica particles R₃ with avidin immobilized thereon. The silica particles R₃ are different from the particles of Comparative Example 2 in terms of the precursor particles.

First, silica particles r₃ manufactured by NIPPN TecnoCluster, Inc were prepared. The particles r₃ had a particle size of 3.0 μm, a specific surface area of 1.2 m²/g and a density of 1.96 g/cm³. 1 g of particles r₃ were dispersed into toluene and then 0.5 g of 1,3,5,7-tetramethylcyclotetrasiloxane (manufactured by Shin-Etsu Chemical Co., Ltd., LS-8600) were added to the resultant dispersion. The dispersion was evaporated to remove toluene, and then the particles were allowed to stand in a vacuum desiccator at 50° C. for 4 hours. Subsequently, the particles were heated in a thermostatic bath at 150° C. for 1.5 hours. It was confirmed that such a treatment made the silica particles r₃ hydrophobic and the creates a coating of 1,3,5,7-tetramethylcyclotetrasiloxane on the silica particles r₃.

Subsequently, the resultant particles were dispersed into water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. The particles were washed, filtered and then dried to obtain silica particles r₃ with a hydroxyl group immobilized on the surface thereof.

Subsequently, an aqueous solution prepared by dissolving 100 mg of avidin in 20 ml of 10 mM PBS solution (pH 7.2) was supplied to 200 mg of the resultant particles, followed by stirring overnight. The particles were washed with a 10 mM PBS solution (pH 7.2) and water and then vacuum-dried to obtain silica particles R₃ with avidin immobilized thereon. The resultant particles R₃ had a specific surface area of 1.2 m²/g, a density of 1.96 g/cm³ and a particle size of about 3.0 μm (a difference between the particle size of the particles R₃ and the particle size of the zirconia particles r₃ is within a measurement deviation).

### Comparative Example 4

Particles prepared in Comparative Example 4 are tungsten particles R₄ with avidin immobilized thereon. The tungsten particles R₄ are different from those of Comparative Examples 2 and 3 in terms of the precursor particles.

First, tungsten particles r₄ manufactured by Hitachi Metals, Ltd. were prepared. The particles r₄ had a particle size of 100 μm, a specific surface area of 0.003 m²/g and a density of 19.1 g/cm³. 1 g of particles r₄ were dispersed into toluene and then 0.5 g of 1,3,5,7-tetramethylcyclotetrasiloxane (manufactured by Shin-Etsu Chemical Co., Ltd., LS-8600) were added to the resultant dispersion. The dispersion was evaporated to remove toluene, and then the particles were allowed to stand in a vacuum desiccator at 50° C. for 4 hours. Subsequently, the particles were heated in a thermostatic bath at 150° C. for 1.5 hours. It was confirmed that such a treatment made the particles r₄ hydrophobic and a coating of 1,3,5,7-tetramethylcyclotetrasiloxane was formed on the particles r₄.

Subsequently, the resultant particles were dispersed into water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. The particles were washed, filtered and then dried to obtain tungsten particles r₄ with a hydroxyl group immobilized on the surface thereof. The particles r₄ were hydrophilic particles.

Subsequently, an aqueous solution prepared by dissolving 100 mg of avidin in 20 ml of 10 mM PBS solution (pH 7.2) was supplied to 200 mg of the resultant particles, followed by stirring overnight. The particles were washed with a 10 mM PBS solution (pH 7.2) and water and then vacuum-dried to obtain tungsten particles R₄ with avidin immobilized thereon. The resultant particles R₄ had a specific surface area of 0.003 m²/g, a density of 19.1 g/cm³ and a particle size of about 100 μm (a difference between the particle size of the particles R₄ and the particle size of the zirconia particles r₄ is within a measurement deviation).

### Comparative Example 5

Particles prepared in Comparative Example 5 are zirconia particles R₅ having a porous structure with avidin immobilized thereon. The zirconia particles R₅ are different from those of Comparative Examples 2 to 4 in terms of the precursor particles.

First, porous zirconia particles (ZirChrom-PHASE) r₅ manufactured by ZirChrom were prepared. The particles r₅ had a particle size of 25 μm, a specific surface area of 30 m²/g and a density of 6 g/cm³. 1 g of particles r₅ was dispersed into toluene and then 0.5 g of 1,3,5,7-tetramethylcyclotetrasiloxane (manufactured by Shin-Etsu Chemical Co., Ltd., LS-8600) was added to the resultant dispersion. The dispersion was evaporated to remove toluene, and then the particles were allowed to stand in a vacuum desiccator at 50° C. for 4 hours. Subsequently, the particles were heated in a thermostatic bath at 150° C. for 1.5 hours. It was confirmed that such a treatment made the particles r₅ hydrophobic and a coating of 1,3,5,7-tetramethylcyclotetrasiloxane was formed on the particles r₅.

Subsequently, the resultant particles were dispersed into water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. The particles were washed, filtered and then dried to obtain porous zirconia particles r₅ with a hydroxyl group immobilized on the surface thereof. The particles r₅ were hydrophilic particles.

Subsequently, an aqueous solution prepared by dissolving 100 mg of avidin in 20 ml of 10 mM PBS solution (pH 7.2) was supplied to 200 mg of the resultant particles, followed by stirring overnight. The particles were washed with a 10 mM PBS solution (pH 7.2) and water and then vacuum-dried to obtain zirconia particles R₅ having a porous structure with avidin immobilized thereon. The resultant particles R₅ had a specific surface area of 30 m²/g, a density of 6 g/cm³ and a particle size of about 25 μm (a difference between the particle size of the particles R₅ and the particle size of the zirconia particles r₅ is within a measurement deviation).

### Comparative Example 6

Particles prepared in Comparative Example 6 are porous silica particles R₆ with avidin immobilized thereon. The porous silica particles R₆ are different from those of Comparative Examples 2 to 5 in terms of the precursor particles.

First, porous silica particles (SUNSPHERE L-121) r₆ manufactured by AGC Si-Tech. Co., Ltd. were prepared. The particles r₆ had a particle size of 11.5 μm, a specific surface area of 336 m²/g and a density of 2.0 g/cm³. 1 g of particles r₆ was dispersed into toluene and then 0.5 g of 1,3,5,7-tetramethylcyclotetrasiloxane (manufactured by Shin-Etsu Chemical Co., Ltd., LS-8600) was added to the resultant dispersion. The dispersion was evaporated to remove toluene, and then the particles were allowed to stand in a vacuum desiccator at 50° C. for 4 hours. Subsequently, the particles were heated in a thermostatic bath at 150° C. for 1.5 hours. It was confirmed that such a treatment made the particles r₆ hydrophobic and a coating of 1,3,5,7-tetramethylcyclotetrasiloxane was formed on the particles r₆.

Subsequently, the resultant particles were dispersed into water and then heated up to 80° C. 10 mg of chloroplatinic acid and 0.5 g of LIGHT-ESTER manufactured by KYOEISHA CHEMICAL Co., LTD. were added to the resultant dispersion, followed by stirring at 80° C. for 4 hours. After washing the particles with water, a dispersion obtained by supplying 10 ml of 10 wt % ethanolamine to the particles was stirred at room temperature for 12 hours. The particles were washed, filtered and then dried to obtain porous silica particles r₆ with avidin immobilized on the surface thereof. The particles r₆ were hydrophilic particles.

Subsequently, an aqueous solution prepared by dissolving 100 mg of avidin in 20 ml of 10 mM PBS solution (pH 7.2) was supplied to 200 mg of the resultant particles, followed by stirring overnight. The particles were washed with a 10 mM PBS solution (pH 7.2) and water and then vacuum-dried to obtain porous silica particles R₆ with avidin immobilized thereon. The resultant particles R₆ had a specific surface area of 336 m²/g, a density of 2.0 g/cm³ and a particle size of about 11.5 μm (a difference between the particle size of the particles R₆ and the particle size of the zirconia particles r₆ is within a measurement deviation).

### Comparative Example 7

Particles prepared in Comparative Example 7 are crosslinked acrylic particles R₇ with an epoxy group immobilized thereon. The crosslinked acrylic particles R₇ are different from those of Comparative Example 1 in terms of a functional group to be immobilized.

First, crosslinked acrylic particles r₇ manufactured by Soken Chemical & Engineering Co., Ltd. were prepared. The particles r₇ had a particle size of 30 μm, a specific surface area of 0.03 m²/g and a density of 1.19 g/cm³. 10 g of particles r₇ were dispersed in 25 g of pure water and 3 g of 3-glycidoxypropyltrimethoxysilane having a terminal epoxy group was added to the resultant dispersion, followed by stirring for 4 hours. Subsequently, the particles were washed, filtered and then dried to obtain crosslinked acrylic particles R₇ with an epoxy group immobilized thereon. The resultant particles R₇ had a specific surface area of 0.03 m²/g, a density of 1.19/cm³ and a particle size of about 30 μm (a difference between the particle size of the particles R₇ and the particle size of the acrylic particles r₇ is within a measurement deviation). The particles R₇ were hydrophilic particles.

### Comparative Example 8

Particles prepared in Comparative Example 8 are porous zeolite particles R₈ with avidin immobilized thereon. The porous zeolite particles R₈ are different from those of Comparative Example 2 in terms of a preparation method therefor.

First, zeolite particles (HSZ-700) r₈ manufactured by TOSOH Corporation were prepared. The particles r₈ had a particle size of 18 μm, a specific surface area of 170 m²/g and a density of 2.3 g/cm³. 10 g of particles r₈ were dispersed into 25 g of pure water and then 3 g of 3-glycidoxypropyltrimethoxysilane having a terminal epoxy group was added to the resultant dispersion, followed by stirring for 4 hours. Subsequently, the particles were washed, filtered and then dried to obtain porous zeolite particles r₈ with an epoxy group immobilized thereon. The particles r₈ were hydrophilic particles. Subsequently, an aqueous solution prepared by dissolving 100 mg of avidin in 20 ml of 10 mM PBS solution (pH 7.2) was supplied to 200 mg of the resultant particles, followed by stirring overnight. The particles were washed with a 10 mM PBS solution (pH 7.2) and water and then vacuum-dried to obtain zirconia particles R₈ with avidin immobilized thereon. The resultant particles R₈ had a specific surface area of 170 m²/g, a density of 2.3 g/cm³ and a particle size of about 18 μm (a difference between the particle size of the particles R₈ and the particle size of the acrylic particles r₈ is within a measurement deviation).

### Comparative Example 9

Particles R₉ prepared in Comparative Example 9 are Dynabeads M-280 Tosylactivated particles with an “anti-human CRP monoclonal antibody 6404” immobilized thereon.

The particles R₉ were prepared by adding an anti-human CRP monoclonal antibody 6404 (manufactured by MedixBiochemica) to Dynabeads M-280 Tosylactivated particles (manufactured by Dynal), followed by stirring, washing through magnetic separation and further immobilization of an anti-human CRP monoclonal antibody 6404 (manufactured by MedixBiochemica) on the surface of the particles. It was confirmed from color development using a HRP-Rabbit-Anti-Mouse IgG2 secondary antibody that the anti-human CRP monoclonal antibody 6404 is immobilized on the surface of the particles. The resultant particles R₉ had a specific surface area of 6 m²/g, a density of 1.3 g/cm³ and a particle size of 2.8 μm.

Various conditions of Examples 1 to 16 and Comparative Examples 1 to 9 are shown in Table 1.

TABLE 1

| | | Particle | | | | |
|---|---|---|---|---|---|---|
| | Particle body | Particle | Specific | | | |
| | Material (precursor | size | surface area | Density | Immobilized substance | |
| | particle) | (μm) | (m²/g) | (g/cm³) | or functional group | Remarks |
| Example 1 | Yttrium-doped zirconia | 50 | 0.02 | 6 | Hydroxyl group | |
| Example 2 | Yttrium-doped zirconia | 50 | 0.02 | 6 | Hydroxyl group | Magnetic particle |
| Example 3 | Yttrium-doped zirconia | 50 | 0.02 | 6 | Hydroxyl group | Production method is different from |
| | | | | | | that of Example 1 |
| Example 4 | Yttrium-doped zirconia | 50 | 0.02 | 6 | Hydroxyl group | Production method is different from |
| | | | | | | those of Examples 1 and 3 |
| Example 5 | Yttrium-doped zirconia | 50 | 0.02 | 6 | Avidin | |
| Example 6 | Alumina | 200 | 0.008 | 3.6 | Avidin | |
| Example 7 | Copper | 50 | 0.013 | 8.9 | Avidin | |
| Example 8 | Yttrium-doped zirconia | 30 | 0.03 | 6 | Avidin | |
| Example 9 | Yttrium-doped zirconia | 15 | 0.04 | 6 | Avidin | |
| Example 10 | Yttrium-doped zirconia | 30 | 0.05 | 5.5 | Epoxy group | Coating polymer of polystyrene |
| Example 11 | Yttrium-doped zirconia | 30 | 0.05 | 5.5 | Avidin | Coating polymer of polystyrene |
| Example 12 | Yttrium-doped zirconia | 30 | 0.05 | 5.5 | Epoxy group | Coating polymer of crosslinked |
| | | | | | | polystyrene |
| Example 13 | Yttrium-doped zirconia | 32 | 0.05 | 6.5 | Epoxy group | Coating polymer of polystyrene |
| | | | | | | Magnetic particle |
| Example 14 | Yttrium-doped zirconia | 50 | 0.02 | 6 | Anti-human CRP | |
| | | | | | monoclonal | |
| | | | | | antibody 6404 | |
| Example 15 | Yttrium-doped zirconia | 50 | 0.02 | 6 | Hydroxyl group | Production method is different from |
| | | | | | | that of Example 1 |
| Example 16 | Yttrium-doped zirconia | 50 | 0.02 | 6 | Avidin | |
| Comparative | Crosslined acrylic | 30 | 0.033 | 1.19 | Hydroxyl group | |
| Example 1 | material | | | | | |
| Comparative | Porous zeolite | 18 | 170 | 2.3 | Avidin | |
| Example 2 | | | | | | |
| Comparative | Silica | 3.0 | 1.2 | 1.96 | Avidin | |
| Example 3 | | | | | | |
| Comparative | Tungsten | 100 | 0.003 | 19.1 | Avidin | |
| Example 4 | | | | | | |
| Comparative | Porous zirconia | 25 | 30 | 6 | Avidin | |
| Example 5 | | | | | | |
| Comparative | Porous silica | 11.5 | 336 | 2.0 | Avidin | |
| Example 6 | | | | | | |
| Comparative | Crosslined acrylic | 30 | 0.03 | 1.19 | Epoxy group | |
| Example 7 | material | | | | | |
| Comparative | Porous zeolite | 18 | 170 | 2.3 | Avidin | Production method is different from |
| Example 8 | | | | | | that of Comparative Example 2 |
| Comparative | Dynabeads M-280 Tosyl- | 2.8 | 6 | 1.3 | Anti-human CRP | |
| Example 9 | activated Particle | | | | monoclonal | |
| | | | | | antibody 6404 | |

Confirmatory Test of Separation Rate of Particles

The tests were carried out so as to confirm a separation rate of particles prepared in Examples and Comparative Examples.

First, particles (1 g) prepared in Examples and Comparative Examples were respectively dispersed in 5 ml of water in a test tube and then allowed to stand. The time required until a transparent supernatant was formed after standing (hereinafter also referred to as “separation time”) was measured. The separation time can be indirectly apprehensible from a movement rate attributable to the spontaneous sedimentation of particles (namely, from spontaneous sedimentation rate of the particles). A similar operation was carried out in a state where a magnet is disposed in the vicinity of the bottom of the test tube. The results of the confirmation test are shown in Table 2.

TABLE 2

| | | Spontaneous |
|---|---|---|
| | Only spontaneous | sedimentation and |
| | sedimentation | magnetic means |
| Example 1 | 20 seconds | 20 seconds |
| Example 2 | 20 seconds | 10 seconds |
| Example 3 | 20 seconds | 20 seconds |
| Example 4 | 20 seconds | 20 seconds |
| Example 5 | 20 seconds | 20 seconds |
| Example 6 | 20 seconds | 20 seconds |
| Example 7 | 20 seconds | 20 seconds |
| Example 8 | 20 seconds | 20 seconds |
| Example 9 | 25 seconds | 25 seconds |
| Example 10 | 40 seconds | 40 seconds |
| Example 11 | 40 seconds | 40 seconds |
| Example 12 | 40 seconds | 40 seconds |
| Example 13 | 40 seconds | 10 seconds |
| Example 14 | 20 seconds | — |
| Example 15 | 20 seconds | 20 seconds |
| Example 16 | 20 seconds | 20 seconds |
| Comparative Example 1 | 100 seconds and more | 100 seconds and more |
| Comparative Example 2 | 80 seconds and more | 80 seconds and more |
| Comparative Example 3 | 90 seconds and more | 90 seconds and more |
| Comparative Example 4 | 15 seconds | 15 seconds |
| Comparative Example 5 | 40 seconds | 40 seconds |
| Comparative Example 6 | 100 seconds and more | 100 seconds and more |
| Comparative Example 7 | 100 seconds and more | 100 seconds and more |
| Comparative Example 8 | 80 seconds and more | 80 seconds and more |
| Comparative Example 9 | — | 60 seconds and more |

From the results shown in Table 2, the following conclusions are drawn:
(a) In general, the spontaneous sedimentation rates in the case of the high-density particles of the Examples are considerably higher than those of Comparative Examples (it should be noted that the sedimentation rate of Comparative Example 4 is due to a high density of 19.1 g/cm³). In other words, the separation rate of the particles of the present invention is higher than that of Comparative Examples. When using the particles of the present invention, a target substance can be separated from a sample more quickly.
(b) When the spontaneous sedimentation rates are compared with respect to particles having a similar density (1.96 g/cm³ to 2.3 g/cm³) of Comparative Examples 2, 3, 6 and 8, the particles having a large specific surface area such as in Comparative Example 6 show the lowest spontaneous sedimentation rate. Considering that the precursor particles of Comparative Example 6 are porous particles, it is presumed that such porous particles with a large specific surface area (namely, the particles having the interpenetrating network structures) have lots of voids, and thereby the air incorporated into the voids inhibits the particles from settling out. That is, a separation rate decreases in the case of the porous particles having a large specific surface area. In this regard, the particles of the present invention have a comparatively small specific surface area and thus the particles of the present invention can separate a target substance from a sample more quickly in light of the small specific surface area.
(c) As compared between the particles of Example 1 and Example 2, or between the particles of Example 10 and Example 13, the particles can be separated more quickly by applying an auxiliary magnetic separation to the magnetic particles in addition to spontaneous sedimentation.

[symbol] Confirmatory Test of Surface State of Precursor Particles

[symbol]

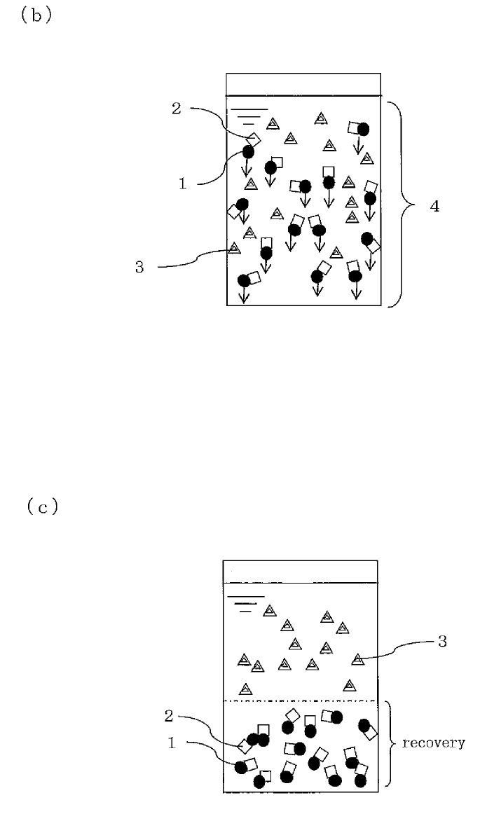

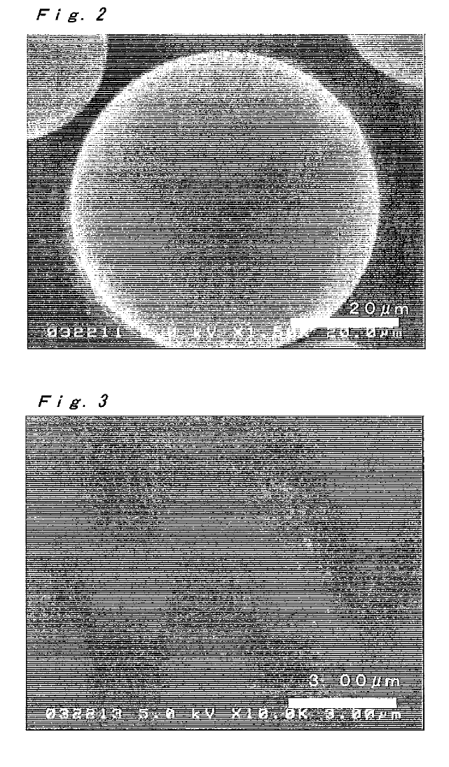

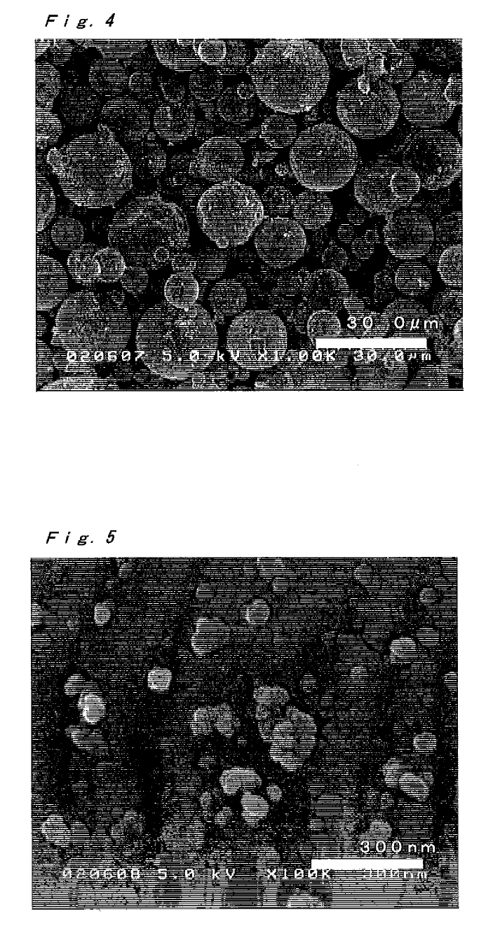

Using a scanning electron microscope (SEM, Model S-4500) manufactured by Hitachi, Ltd., a surface state of the precursor particles used in Example 1 and Comparative Example 6 was observed. The results are shown in FIG. 2 to FIG. 5. FIG. 2 and FIG. 3 are electron micrographs showing yttrium-doped zirconia particles p₁ used in Example 1, whereas FIG. 4 and FIG. 5 are electron micrographs showing porous silica particles r₆ used in Comparative Example 6. As is apparent from FIG. 2 to FIG. 5, the particle of Example 1 is non-porous particle and the surface of the particle is free from roughness so that the surface thereof is smooth, whereas, the particle of Comparative Example 6 is porous particle and the surface of the particle has large roughness. As a result, it can be understood that these particles significantly differ in a specific surface area, that is, the particle of the present invention has a smaller specific surface area. Referring to FIG. 2 and FIG. 3, it can be understood that “particle body has no through-pore” in a case of the particle of the present invention.

[symbol] Confirmatory Test of Specific/Nonspecific Binding Characteristics of Particles

[symbol]

Using particles P₅, particles P₁₁ and particles P₁₆ prepared in Examples 5, 11 and 16 as well as particles R₂, particles R₄, particles R₅, particles R₆ and particles R₈ prepared in Comparative Example 2, 4, 5, 6 and 8, specific/nonspecific binding characteristics of the particles (P₅, P₁₁, P₁₆, R₂, R₄, R₅, R₆ and R₈) were confirmed. As a target substance, two kinds of substances, i.e. HRP and biotinylated HRP were used (enzyme activities of both substances are almost the same). Avidin immobilized on the particles is capable of specifically binding to biotinylated HRP, but is not capable of specifically binding to HRP. That is, biotinylated HRP can specifically (preferentially) bind to the particles, whereas HRP can nonspecifically bind to the particles due to its adsorption into pore-containing region of the particles.

Since the same operation was applied to particles P₅, P₁₁ and P₁₆, as well as the particles R₂, R₄, R₅, R₆ and R₈, an operation for particles P₅ of Example 5 will be mainly described. First, two 1.5 ml tubes were prepared and an appropriate amount (which enables a chromogenic amount of 0.01 to 1.5) of particles P₅ were respectively charged thereinto. 100 μl of biotinylated HRP having a concentration of 20 ng/ml was added to one tube whereas 100 μl of HRP having a concentration of 20 ng/ml was added to the other tube, followed by stirring respectively for 30 minutes using a vortex mixer. The particles P₅ charged in each tube was washed with 400 μl of a 10 mM PBS buffer solution (pH 7.2) and then subjected to centrifugal separation. This washing and centrifugal separation were carried out four times. A PBS buffer solution (pH 7.2) was removed and 200 μl of TMB (tetramethylbenzidine) was added to each tube containing the particles P₅, followed by standing for 30 minutes, and thereby causing color development of the particles P₅. The reaction was terminated by adding 200 μl of 1N sulfuric acid. The amount of light emitted from the particles P₅ charged in each tube was determined by measuring an absorbance (450 nm) using Microplate Reader Infinite 200 manufactured by TECAN. The amount of light emitted from the particles P₅ to which biotinylated HRP was provided is proportional to the amount of biotinylated HRP which has specifically bound to the particles P₅. The amount of light emitted from the particles P₅ to which HRP was provided is proportional to the amount of HRP which has nonspecifically bound to the particles P₅. Therefore, when a ratio (I_{specific}/I_{nonspecific}) of the chromogenic amount I_{specific} for the specifically bound biotinylated HRP to the chromogenic amount I_{nonspecific} of nonspecifically bound HRP is high, the particles exhibit slighter nonspecific binding characteristics. In contrast, when the ratio (I_{specific}/I_{nonspecific}) is small, the particles exhibit greater nonspecific binding characteristics.

A similar operation was applied to particles P₁₁ of Example 11, particles P₁₆ of Example 16, particles R₂ of Comparative Example 2, particles R₄ of Comparative Example 4, particles R₅ of Comparative Example 5, particles R₆ of Comparative Example 6 and particles R₈ of Comparative Example 8, and then the chromogenic amount I_{specific} for the biotinylated HRP which has specifically bound to the particles and the chromogenic amount I_{nonspecific} for the HRP which has nonspecifically bound to the particles were determined.

The results are shown in Table 3. As is apparent from the results shown in Table 3, particles P₅ of Example 5, particles P₁₁ of Example 11 and particles P₁₆ of Example 16 show higher values of I_{specific}/I_{nonspecific} when compared with particles R₂ of Comparative Example 2, particles R₅ of Comparative Example 5, particles R₆ of Comparative Example 6 and particles R₈ of Comparative Example 8. That is, nonspecific binding is significantly suppressed in the case of particles P₅, P₁₁ and P₁₆. It can be therefore understood that the particles of the present invention, which have a small specific surface area and thus are substantially non-porous particles, can suppress the nonspecific binding phenomenon in which “substances other than a target substance” bind to the particles. The chromogenic amount for “nonspecific” (I_{nonspecific}) of Comparative Example 4 is small due to the non-porosity of the particles R₄, but the chromogenic amount (I_{specific}) for “specific adsorption” is also small. The reason for this is not clear, but is presumed that the precursor particles are involved. Specifically, it is presumed that, when avidin was provided on the precursor particles by performing a surface treatment, the avidin was broken by the precursor particles due to the too high specific gravity of the particles, which led to an inability of the specifically-binding characteristic of the biotinylated HRP.

TABLE 3

| | Amount I_{specific} of light emitted | Amount I_{nonspecific} of light emitted | |
|---|---|---|---|
| | from biotinylated HRP which has | from HRP which has nonspecifically | |
| | specifically bound to the particles | bound to the particles | I_{specific}/I_{nonspecific} |
| Particles P₅ of | 0.11 | 0.03 | 3.5 |
| Example 5 | | | |
| Particles P₁₁ of | 0.14 | 0.035 | 4.0 |
| Example 11 | | | |
| Particles P₁₆ of | 0.13 | 0.033 | 3.9 |
| Example 16 | | | |
| Particles R₂ of | 0.13 | 0.065 | 2.0 |
| Comparative Example 2 | | | |
| Particles R₄ of | 0.05 | 0.03 | 1.5 |
| Comparative Example 4 | | | |
| Particles R₅ of | 0.10 | 0.061 | 1.6 |
| Comparative Example 5 | | | |
| Particles R₆ of | 0.095 | 0.068 | 1.4 |
| Comparative Example 6 | | | |
| Particles R₈ of | 0.099 | 0.065 | 1.5 |
| Comparative Example 8 | | | |

### CONCLUSION

As is apparent from the results of the above “confirmatory test of separation rate of particles” and “confirmatory test of specific/nonspecific binding characteristics of particles”, the particles of the present invention are capable of providing a sufficient separation rate only by a movement rate resulting from spontaneous sedimentation thereof as well as suppressing the nonspecific binding phenomenon in which “substances other than a target substance” bind to the particles. It could be confirmed from the “confirmatory test of a surface state of precursor particles” that the particles of the present invention are non-porous particles and thus “particle body has no through-pore”.

1. A particle to which a target substance can bind, characterized in that:
a substance or functional group capable of binding to the target substance is immobilized on a surface of a particle body;
a density of the particle is in the range of 3.5 g/cm³ to 9.0 g/cm³; and
the particle body has no through-pore.

2. The particle according to claim 1, characterized in that a specific surface area of the particle is in the range of 0.0005 m²/g to 1.0 m²/g.

3. The particle according to claim 1, characterized in that a coating of polymer is provided on a part of the surface of the particle body; and
the substance or functional group capable of binding to the target substance is immobilized on a surface of the particle body or polymer.

4. The particle according to claim 3, characterized in that the coating of polymer is provided over an entire surface of the particle body; and
the substance or functional group capable of binding to the target substance is immobilized on the surface of the polymer.

5. The particle according to claim 3, characterized in that the polymer is at least one kind of a polymer selected from the group consisting of polystyrene, poly(meth)acrylic acid, poly(meth)acrylic acid ester, polyvinylether, polyurethane, polyamide, polyvinyl acetate, polyvinyl alcohol, polyallylamine and polyethyleneimine.

6. The particle according to claim 3, characterized in that the polymer is a crosslinked polymer.

7. The particle according to claim 1, characterized in that the particle is a non-porous particle.

8. The particle according to claim 1, characterized in that the particle body is made of at least one kind of a material selected from the group consisting of zirconia, yttrium-doped zirconia, iron oxide and alumina.

9. The particle according to claim 1, characterized in that the particle is a magnetic particle.

10. The particle according to claim 9, characterized in that a saturation magnetization is in the range of 0.5 to 85 A·m²/kg.

11. The particle according to claim 1, characterized in that an average size of the particle is in the range of 1 μm to 1 mm.

12. The particle according to claim 1, characterized in that the substance capable of binding to the target substance is at least one kind of a substance selected from the group consisting of biotin, avidin, streptavidin and neutravidin.

13. The particle according to claim 1, characterized in that the functional group capable of binding to the target substance is at least one kind of a functional group selected from the group consisting of carboxyl group, hydroxyl group, epoxy group, tosyl group, succinimide group, maleimide group, thiol group, thioether group, disulfide group, aldehyde group, azido group, hydrazide group, primary amino group, secondary amino group, tertiary amino group, imide ester group, carbodiimide group, isocyanate group, iodoacetyl group, halogen-substitution of carboxyl group and double bond.

14. The particle according to claim 3, characterized in a silicon-containing substance and/or polyethylene glycol is present on at least a part of the surface of the particle body and/or the surface of the polymer.

15. The particle according to claim 1, characterized in that the target substance can bind to the particle by an adsorptivity or affinity generated between the target substance and the substance or functional group capable of binding to the target substance.

16. The particle according to claim 15, characterized in that the affinity generated between the target substance and the substance or functional group capable of binding to the target substance is due to an electrostatic interaction, π-π interaction, π-cation interaction, dipolar interaction, hydrophobic interaction, hydrogen bond, coordinate bond or biochemical interaction.

17. A method for separating a target substance from a sample or obtaining a particle with a target substance immobilized thereon, by the use of the particle according to claim 1, the method comprising the steps of:
(i) bringing the particle and the sample containing the target substance into contact with each other, and thereby binding the particle and the target substance to each other,
(ii) allowing the sample to stand, and thereby allowing a spontaneous sedimentation of the particle in the sample, and
(iii) recovering the particle precipitated in the sample, and thereby separating the target substance from the sample or obtaining the particle with the target substance immobilized thereon.

18. A method for separating a target substance from a sample or obtaining a particle with a target substance immobilized thereon, by the use of the particle according to claim 2, the method comprising the steps of:
(i) bringing the particle and the sample containing the target substance into contact with each other, and thereby binding the particle and the target substance to each other,
(ii) allowing the sample to stand, and thereby allowing a spontaneous sedimentation of the particle in the sample, and
(iii) recovering the particle precipitated in the sample, and thereby separating the target substance from the sample or obtaining the particle with the target substance immobilized thereon.

19. A method for separating a target substance from a sample or obtaining a particle with a target substance immobilized thereon, by the use of the particle according to claim 3, the method comprising the steps of:
(i) bringing the particle and the sample containing the target substance into contact with each other, and thereby binding the particle and the target substance to each other,
(ii) allowing the sample to stand, and thereby allowing a spontaneous sedimentation of the particle in the sample, and
(iii) recovering the particle precipitated in the sample, and thereby separating the target substance from the sample or obtaining the particle with the target substance immobilized thereon.

20. A method for performing an analysis, extraction, purification or reaction of a target substance by utilization of the method according to claim 17.
